(19) United States
(12) Patent Application Publication
(10) Pub. No.: US 2024/0296833 A1
(43) Pub. Date: Sep. 5, 2024

(54) WEARABLE SILENT SPEECH DEVICE, SYSTEMS, AND METHODS FOR ADJUSTING A MACHINE LEARNING MODEL

(71) Applicants: Sahaj Garg, Foster City, CA (US); Anthony Leonardo, Broadlands, VA (US); Tanay Kothari, San Francisco, CA (US)

(72) Inventors: Sahaj Garg, Foster City, CA (US); Anthony Leonardo, Broadlands, VA (US); Tanay Kothari, San Francisco, CA (US)

(21) Appl. No.: 18/648,138

(22) Filed: Apr. 26, 2024

Related U.S. Application Data
(63) Continuation-in-part of application No. 18/338,827, filed on Jun. 21, 2023.
(60) Provisional application No. 63/437,088, filed on Jan. 4, 2023.

(51) Int. Cl. G10L 15/06; A61B 5/389; G10L 15/24; G10L 25/78

(52) U.S. Cl. CPC G10L 15/063; A61B 5/389; G10L 15/24; G10L 25/78

(57) ABSTRACT

The present disclosure relates to methods and systems for adjusting a silent speech machine learning model for use with a wearable silent speech device. In some embodiments, a method may include recording speech signals from a user, using a first sensor and a second sensor of a wearable silent speech device. The method may include providing for a silent speech machine learning model for use with the wearable silent speech device, determining whether the silent speech machine learning model is to be adjusted, and in response to determining the silent speech machine learning model is to be adjusted, adjusting the silent speech machine learning model based on at least the speech signals recorded using the first sensor and the second sensor.

### CROSS-REFERENCE TO RELATED APPLICATIONS

This application is a continuation in-part of U.S. patent application Ser. No. 18/338,827, titled “WEARABLE SILENT SPEECH DEVICE, SYSTEMS, AND METHODS”, filed Jun. 21, 2023, which claims the benefit under 35 U.S.C. § 119(c) of U.S. Provisional Application No. 63/437,088, entitled “SYSTEM AND METHOD FOR SILENT SPEECH DECODING,” filed Jan. 4, 2023, the entire contents of both of which are incorporated herein by reference for all purposes.

### BACKGROUND

Traditional communication modalities and interactive systems require user inputs including voiced speech, typing and/or selection of various system inputs during use. Many of these interactive systems use various input methods and devices, such as microphones, keyboard/mouse devices and other devices and methods for receiving inputs from users. It is often desirable for these systems to remain accurate over extended periods of time and across different user conditions.

### SUMMARY

According to some embodiments, a method for adjusting a silent speech machine learning model is provided. The method includes: recording speech signals from a user, using a first sensor and a second sensor of a wearable silent speech device; providing for a silent speech machine learning model for use with the wearable silent speech device; determining whether the silent speech machine learning model is to be adjusted; and in response to determining the silent speech machine learning model is to be adjusted, adjusting the silent speech machine learning model based on at least the speech signals recorded using the first sensor and the second sensor.

According to any of the above embodiments, the method further includes determining a subset of the recorded speech signals, and adjusting the silent speech machine learning model comprises: providing the subset of the recorded speech signals to a silent speech machine learning model; conditioning the silent speech machine learning model based on the subset of the recorded speech signals; and processing, using the conditioned silent speech machine learning model, the recorded speech signals to generate a representation of one or more words spoken by the user.

According to any of the above embodiments, the method further includes storing the recorded speech signals in non-volatile storage, the non-volatile storage storing historic speech signals recorded by the wearable silent speech device, and adjusting the silent speech model involves training the silent speech machine learning model based on the speech signals recorded using the first sensor and the second sensor and the historic speech signals.

According to any of the above embodiments, the first sensor is an EMG sensor and the second sensor is a microphone.

According to any of the above embodiments, the determining includes determining whether the recorded speech signals are suitable for use in adjusting the silent speech machine learning model, and determining the silent speech machine learning model is to be adjusted in response to determining the recorded speech signals are suitable for use in adjusting the silent speech machine learning model.

According to any of the above embodiments, determining whether the recorded speech signals are suitable includes determining, based on the speech signals, whether the user is speaking out loud and determining the recorded speech signals are suitable in response to determining the user is speaking out loud.

According to any of the above embodiments, determining whether the recorded speech signals are suitable includes determining, based on the speech signals, whether the user is whispering and determining the recorded speech signals are suitable in response to determining the user is whispering.

According to any of the above embodiments, determining whether the recorded speech signals are suitable includes determining, based on the speech signals, whether the user is speaking silently, and determining the recorded speech signals are suitable in response to determining the user is speaking silently.

According to any of the above embodiments, determining whether the recorded speech signals are suitable includes determining, based on the speech signals, a level of background noise and determining the recorded speech signals are suitable in response to determining the level of background noise is below a threshold level.

According to any of the above embodiments, determining whether the recorded speech signals are suitable includes determining whether the speech signals are associated with the user communicating with the wearable silent speech device, and determining the recorded speech signals are suitable in response to determining the user is communicating with the wearable silent speech device.

According to any of the above embodiments, determining whether the recorded speech signals are suitable includes determining whether the speech signals are associated with the user communicating with the wearable silent speech device, and determining the recorded speech signals are suitable in response to determining the user is not communicating with the wearable silent speech device.

According to any of the above embodiments, determining whether the silent speech machine learning model is to be adjusted includes determining whether the silent speech machine learning model requires user onboarding, and in response to determining the silent speech machine learning model requires user onboarding, prompting the user to speak one or more words or phrases, wherein the speech signals are recorded after the prompting.

According to any of the above embodiments, determining whether the silent speech machine learning model is to be adjusted includes determining a performance metric of the silent speech machine learning model; and determining the silent speech machine learning model is to be adjusted in response to determining the performance metric exceeds a threshold level.

According to any of the above embodiments, determining whether the silent speech machine learning model is to be adjusted includes determining, based on a user input, whether the silent speech machine learning model is to be adjusted.

According to any of the above embodiments, the method further includes determining whether the wearable silent speech device is being powered on, and in response to determining the wearable silent speech device is being powered on, prompting the user to speak one or more words or phrases, wherein the speech signals are recorded after the prompting, and it is determined that the silent speech machine learning model is to be adjusted in response to determining the wearable silent speech device is being powered on.

According to any of the above embodiments, determining whether the silent speech machine learning model is to be adjusted includes determining a time since a last silent speech machine learning model adjustment, and in response to determining the time is above a threshold time, determining the silent speech machine learning model is to be adjusted.

According to any of the above embodiments, the method further includes analyzing the recorded speech signals; and selecting a subset of the recorded speech signals, wherein the adjusting is performed using the subset of the recorded speech signals.

According to any of the above embodiments, analyzing the recorded speech signals includes determining phonetic content of the recorded speech signals; and the subset of recorded speech signals is phonetically balanced.

According to any of the above embodiments, adjusting the silent speech machine learning model includes performing a gradient step of the silent speech machine learning model.

According to any of the above embodiments, the gradient step is performed based on a comparison of an output of the silent speech machine learning model to ground truth data.

According to any of the above embodiments, the method further includes determining the ground truth data based on the recorded speech signals.

According to any of the above embodiments, the ground truth data is determined based on known words or phrases associated with the recorded speech signals.

According to any of the above embodiments, the ground truth data is determined using a second machine learning model, different from the silent speech machine learning model.

According to any of the above embodiments, the silent speech machine learning model is an autoregressive model.

According to any of the above embodiments, the subset of the recorded speech signals are the first three seconds of the recorded speech signals.

According to any of the above embodiments, conditioning the silent speech machine learning model includes generating an embedding of features of the speech signals and conditioning the silent speech machine learning model using the embedding.

According to any of the above embodiments, training the silent speech machine learning model includes performing a series of gradient steps based on a comparison of an output of the silent speech machine learning model to ground truth data.

According to any of the above embodiments, the non-volatile storage stores ground truth data associated with the historic speech signals.

According to any of the above embodiments, the non-volatile storage stores simulated data associated with the first and second sensors and ground truth data associated with the simulated data, and wherein training the silent speech machine learning model is performed based on the speech signals recorded using the first sensor, the second sensor and the historic speech signals, and the simulated data.

According to some embodiments, a system for recognizing silent speech of a user is provided. The system includes: a wearable silent speech device; at least one computer hardware processor; and at least one non-transitory computer-readable storage medium storing processor-executable instructions that, when executed by the at least one computer hardware processor, cause the at least one computer hardware processor to perform a method, the method including: obtaining speech signals recorded from a user, using a first sensor and a second sensor of the wearable silent speech device; providing for a silent speech machine learning model for use with the wearable silent speech device; determining whether the silent speech machine learning model is to be adjusted; and in response to determining the silent speech machine learning model is to be adjusted, adjusting the silent speech machine learning model based on at least the speech signals recorded using the first sensor and the second sensor.

According to some embodiments, at least one non-transitory computer-readable storage medium is provided. The storage medium storing processor-executable instructions that, when executed by at least one computer hardware processor, cause the at least one computer hardware processor to perform a method, the method including: obtaining speech signals recorded from a user, using a first sensor and a second sensor of a wearable silent speech device; providing for a silent speech machine learning model for use with the wearable silent speech device; determining whether the silent speech machine learning model is to be adjusted; and in response to determining the silent speech machine learning model is to be adjusted, adjusting the silent speech machine learning model based on at least the speech signals recorded using the first sensor and the second sensor.

### BRIEF DESCRIPTION OF FIGURES

Various aspects of at least one embodiment are discussed herein with reference to the accompanying figures, which are not intended to be drawn to scale. The figures are included to provide illustration and a further understanding of the various aspects and embodiments and are incorporated in and constitute a part of this specification but are not intended as a definition of the limits of the invention. Where technical features in the figures, detailed description or any claim are followed by reference signs, the reference signs have been included for the sole purpose of increasing the intelligibility of the figures, detailed description, and/or claims. Accordingly, neither the reference signs nor their absence is intended to have any limiting effect on the scope of any claim elements. In the figures, each identical or nearly identical component that is illustrated in various figures is represented by a like numeral. For purposes of clarity, not every component may be labeled in every figure. In the figures:

FIG. 1A depicts a view of a user 101 wearing an embodiment of a wearable device 100, in accordance with some embodiments of the technology described herein.

FIG. 1B is an illustration of wearable device target zone(s) associated with a wearable speech input device such as wearable device 100 (FIG. 1A), in accordance with some embodiments of the technology described herein.

FIG. 2 is a block diagram of a silent speech system 200, in accordance with some embodiments of the technology described herein.

FIG. 3 is an example process flow for adjusting a machine learning model, according to some embodiments of the technology described herein.

FIG. 4A shows an example data flow for performing calibration of a silent speech machine learning model, according to embodiments of the technology described herein.

FIG. 4B shows an example data flow 410 for performing training of a silent speech machine learning model according to some embodiments of the technology described herein.

FIG. 4C shows an example data flow 420 for performing calibration and training of a silent speech machine learning model, according to some embodiments of the technology described herein.

FIG. 4D illustrates an example process flow for adjusting a silent speech machine learning model, according to some embodiments of the technology described herein.

FIG. 5A is an example of performing calibration of the silent speech machine learning model using outputs of the silent speech machine learning model and a second machine learning model, according to some embodiments of the technology described herein.

FIG. 5B illustrates an example data flow 520 for adjusting a silent speech machine learning model using signals recorded by sensors of a wearable silent speech device, according to some embodiments of the technology described herein.

FIG. 5C illustrates an example data flow 530 for adjusting a silent speech machine learning model, according to some embodiments of the technology described herein.

FIG. 5D illustrates an example data flow 540 for adjusting an autoregressive silent speech machine learning model, according to some embodiments of the technology described herein.

FIG. 6A provides an example of performing calibration of a silent speech machine learning model in response to a user speaking out loud, according to some embodiments of the technology described herein.

FIG. 6B provides an example of performing calibration of a silent speech machine learning model 617 in response to a user 601 speaking one or more known words, according to some embodiments of the technology described herein.

FIG. 6C provides an example of performing calibration of a silent speech machine learning model based on a user-initiated calibration according to some embodiments of the technology described herein.

FIG. 6D is an example of performing calibration of a silent speech machine learning model in response to poor performance of the model, in accordance with aspects of the technology described herein.

FIG. 7 illustrates an example of a process for performing training of a silent speech machine learning mode, according to some embodiments of the technology described herein.

FIG. 8A is a scheme diagram of an example speech input device 800 capable of communicating with machine learning models 850 external to the speech input device, according to some embodiments of the technology described herein.

FIG. 8B is a flow diagram of an example process 860 which may be performed by a speech input device such as speech input device 800 shown in FIG. 8A, according to some embodiments of the technology described herein.

FIG. 9A is a scheme diagram of an example speech input device 900 including a silent speech model, according to some embodiments of the technology described herein.

FIG. 9B is a flow diagram of an example process 960 including the use of a silent speech model, where the process may be performed by a speech input device, e.g., 900 (FIG. 9A) according to some embodiments of the technology described herein.

FIG. 10A is a scheme diagram of a machine learning model configured to decode speech to predict text or encoded features using EMG signals, according to some embodiments of the technology described herein.

FIG. 10B is a scheme diagram of a machine learning model 1014 configured to decode speech to predict text or encoded features using EMG signals and segmentation of the EMG signals, according to some embodiments of the technology described herein.

### DETAILED DESCRIPTION

To solve the above-described technical problems and/or other technical problems, the inventors have recognized and appreciated that silent speech or sub-vocalized speech may be particularly useful in communication and may be implemented in in interactive systems. In these systems, for example, users may talk to the system via silent speech or whisper to the system in a low voice for the purposes for providing input to the system and/or controlling the system, without the drawbacks associated with voice-based systems. It is important for such systems to be available to users over extended periods of time in order to allow for effective and continued use and communication during changing conditions.

In at least some embodiments as discussed herein, silent speech is speech in which the speaker does not vocalize their words out loud, but instead mouths the words as if they were speaking with vocalization. In some examples, silent speech may refer to unvoiced mode of phonation in which the vocal cords are abducted so that they do not vibrate, and no audible turbulence is created during speech. Silent speech may occur at least in part while the user is inhaling, and/or exhaling. Silent speech may occur in a minimally articulated manner, for example, with visible movement of the speech articulator muscles, or with limited to no visible movement, even if some muscles such as the tongue are contracting. In a non-limiting example, silent speech has a volume below a volume threshold (e.g., 30 dB when measured about 10 cm from the user's mouth). In some examples, whispered speech may refer to unvoiced mode of phonation in which the vocal cords are abducted so that they do not vibrate, where air passes between the arytenoid cartilages to create audible turbulence during speech. In some embodiments, as discussed herein, silent speech may include the speaker speaking without vocalization, whispering and/or speaking softly.

As described herein, voiced (vocal) speech may refer to a vocal mode of phonation in which the vocal cords vibrate during at least part of the speech for vocal phonemes, creating audible turbulence during speech. In a non-limiting example, vocal speech may have a volume above a volume threshold (e.g., 40 dB when measured 10 cm from the user's mouth).

Accordingly, the inventors have developed new technologies for silent speech devices allowing the devices to automatically adapt to the current actions of the wearer, allowing for continued interaction with mobile devices, smart devices, communication systems and interactive systems. In some embodiments, the techniques may include a wearable device configured to recognize speech signals of a user including electrical signals indicative of a user's facial muscle movement when the user is speaking (e.g., silently or with voice), motion signals associated with the movement of a wearer's face, vibration signals associated with voiced speech, and/or audio signals, and change its operation in response to the signals. In some examples, the wearable device may additionally or alternatively measure a position of a user's tongue, blood flow of the user, muscle strain of the user, muscle frequencies of the user, temperatures of the user, and magnetic fields of the user, among other signals. Any such signal may be used with the technologies described herein.

In some examples, signals recorded from a wearable silent speech device may be analyzed using one or more machine learning (ML) models. Examples of machine learning models which may analyze the signals include: statistical models, neural networks, and autoregressive machine learning models, flow matching machine learning models, diffusion machine learning models, among other types of machine learning models. In some examples, the machine learning models may output representations of words or phrases spoken, either silently or voiced, by the user, such as in a transcription of the words or phrases, or as an encoded signal representing the words or phrases.

In some examples, the machine learning models which analyze signals from the wearable silent speech device are maintained on the wearable silent speech device. In some examples, the machine learning models are maintained on an external device and analyze recorded speech signals transmitted to the external device. In some examples, the machine learning models are maintained on a server and analyze recorded speech signals transmitted either directly from the wearable silent speech device or indirectly through an external device.

In some examples the machine learning models may be adjusted to better account for characteristics of a user of a wearable silent speech device. In some examples different types of adjustments may be performed. In some examples, model adjustments may be performed continuously as the user is wearing and/or using the wearable silent speech device. In some examples, model adjustments may be performed at set intervals when the user is wearing or using the wearable silent speech device, such as time intervals, for example every minute, two minutes, 3 minutes, 4 minutes, 5 minutes, 5-10 minutes, or 10-20 minutes, or word intervals, for example every 100 words, 500 words, 1,000 words, 2,500 words, 5,000 words, or 10,000 words.

In some examples, adjusting the machine learning model may involve performing one or more training processes including a gradient descent based on the signals recorded by the wearable silent speech device, regression analysis based on the signals recorded by the wearable silent speech device, performing stochastic gradient descent based on the signals recorded by the wearable silent speech device, low-rank adaptation of the model, or momentum training based on the signals recorded by the wearable silent speech device, among other suitable techniques for adjusting a machine learning model. In some examples, the machine learning model may be adjusted through autoregressive generation.

In some examples adjusting a machine learning model, such as a silent speech machine learning model may involve tagging recorded silent speech signals with associated data. In some examples, the recorded data may be tagged with ground truth data. In some examples, ground truth data may be recorded from one or more sensors, for example: audio, video, motion data, and/or any sensor data captured from other measurement modalities. In some examples, the signals recorded as ground truth data may undergo processing before use in training a machine learning model. In some examples, ground truth audio signals (e.g., captured from a microphone or a video camera) may be converted to a text speech label (e.g., using ASR or converted manually). In other examples, ground truth videos may be converted to a text speech label (e.g., using automated lip reading or converted manually). For example, facial kinematics may be extracted from a ground truth video of a training subject when speaking during the training data collection. Lip reading may use the extracted facial kinematics to convert the video to a text speech label.

In some examples, the ground truth data is the output of a second machine learning model, different from the silent speech machine learning model, for example a speech machine learning model. In some examples, the second machine learning model is an automatic speech recognition model, for example OpenAi Whisper. In some examples the output of the second machine learning model is determined based on signals recorded from sensors different from the sensors used to record input signals for the silent speech machine learning model. In some examples the ground truth data is a recorded signal, from a sensor different from those used to record input signals for the silent speech machine learning model, for example an audio signal from a microphone. In some examples, the ground truth data is in a signal format different from the recorded signals. In some examples, the ground truth data is a known signal determined based on the recorded signals.

In some examples, the output of a silent speech machine learning model may be tagged with associated data of a second machine learning model output from signals recorded simultaneously with the silent speech signals. In some examples, adjusting the silent speech machine learning model may be performed based on the tagged output of the silent speech model. In some examples, the silent second machine learning model output and speech machine learning model output may include representations of one or more words spoken by the user and tagging the silent speech machine learning model output may involve tagging each word representation of the silent speech machine learning model output with the associated word representation of the second machine learning model output. In some examples, the silent speech machine learning model output and second machine learning model output do not contain representations of words spoken by the users, and tagging the silent speech machine learning model output may involve associating portions of the silent speech machine learning model output with portions of the second learning model output. For example, the tagging may involve temporally aligning the silent speech machine learning model output with the second machine learning model output, or aligning the model outputs in any suitable way, for example by frequency. The tagged silent speech machine learning model output may be used to determine model adjustments, which are implemented in the silent speech machine learning model.

In some embodiments, training data for the machine learning model may be associated with a source domain. In some embodiments, the source domain may be a voiced domain, where the signals indicating the user's speech muscle activation patterns are collected from voiced speech of the user. In some embodiments, the source domain may be a whispered domain, where the signals indicating the user's speech muscle activation patterns are collected from whispered speech of the user. In some embodiments, the source domain may be a silent domain, where the signals indicating the user's speech muscle activation patterns are collected from silent speech of the user.

In some examples, adjusting a silent speech machine learning model may involve determining a loss function value based on the silent speech machine learning model output and ground truth data such as the output of a second machine learning model, a known signal, or other recorded signals, among other ground truth data sources described herein. Examples of loss functions which may be used to adjust a silent speech machine learning model include a mean square error, a mean absolute error, a mean bias error, a Huber Loss, a log-cosh loss, a quantile loss, a cross entropy loss, sequence to sequence, masked infilling loss, and Connectionist Temporal Classification (CTC) loss, among other loss functions. In some examples, the loss function value is determined by using the ground truth data and the silent speech machine learning model output as a prediction value for calculating the loss function value. In some examples, the loss function value is determined by using the prediction from a second machine learning model as the label and the silent speech machine learning model output as a prediction value for calculating the loss function value. In some examples, the loss function value is used to perform gradient descent with respect to one or more weights of the silent speech machine learning model. One or more weight adjustments may be determined by performing gradient descent based on the loss function value. The weight adjustments may be provided to the silent speech machine learning model as model adjustments.

In some examples, recorded speech signals may be processed before use in adjusting a machine learning model. For example, signals may be processed by one or more machine learning models, tagged, aligned, cropped, normalized, phonetically balanced, or may undergo other processing as described herein.

In some examples, adjusting the silent speech machine learning model may involve performing calibration of the silent speech machine learning model. In some examples, calibration may involve an adjustment of the silent speech machine learning model to account for a current user state, position of the wearable silent speech device, among other factors. In some examples, calibrations of the silent speech machine learning model are performed continuously as a user is wearing the wearable silent speech device. In some examples, a calibration is a temporary adjustment to the silent speech machine learning model. In some examples, changes to the silent speech machine learning model due to a calibration are overwritten by changes from a subsequent calibration.

In some examples, performing a calibration of the silent speech machine learning model involves performing a training iteration of the silent speech machine learning model based on the signals recorded by the wearable silent speech device, using the training methods described herein. In some examples, a training iteration of the silent speech machine learning model involves performing a single training epoch of the silent speech machine learning model using signals recorded using the sensors of the wearable silent speech device. For example, adjusting the silent speech machine learning model may involve performing a gradient step of the machine learning model, by performing a gradient descent analysis based on the signals recorded by the wearable silent speech device.

In some examples, when a calibration is performed, the quality of the calibration may be validated based upon one or more validation metrics. For example, the gradient norm of the calibration may be analyzed. In some examples, in response to determining the gradient norm is above a threshold level, the calibration is not applied to the silent speech machine learning model. In some examples, if it is determined that the quality of a calibration is poor, the silent speech machine learning model may be reverted to a previous calibration. In some examples, if it is determined that the quality of a calibration is poor, a new calibration may be triggered.

In some examples, performing a calibration involves analyzing the recorded speech signals from the user and selecting a machine learning model from multiple machine learning models for use. In some examples, the selected machine learning model may provide a most accurate estimation of user speech, as described herein. The selected machine learning model may be selected based on one or more features determined from the recorded speech signals or by a comparison of outputs of the multiple machine learning models. In some examples, a machine learning model may be selected based on if the user is speaking out loud, whispering or silently speaking.

In some examples, when recorded speech signals are used for performing a training iteration of the silent speech machine learning model, the recorded speech signals are not maintained in storage of the wearable silent speech device after the training iteration and are replaced by newly recorded speech signals. In some examples, the silent speech machine learning model is continuously adjusted with training iterations based on recorded signals from the wearable silent speech device, when the wearable silent speech device is in use.

In some examples, adjusting the silent speech machine learning model may involve performing training the silent speech machine learning model. Training of the silent speech machine learning model may require more intensive processing than calibration of the silent speech machine learning model. In some examples, training may be used to better tailor the model to the user of the wearable silent speech device. In some examples, adjustments to the silent speech machine learning model due to training are overwritten by changes from subsequent training. In some examples, preforming training of the silent speech machine learning model may involve training the machine learning model using the signals recorded from the sensors of the wearable silent speech device, by performing a training process described herein. In some examples, the signals recorded from the sensors of the wearable silent speech device may be combined with previously recorded data to create a training dataset for training the silent speech machine learning model. In some examples, the previously recorded data includes signals previously recorded from the user by the sensors of the wearable silent speech device. In some examples, the previously recorded data includes general data not recorded from the user. In some examples, performing training the silent speech machine learning model may involve performing multiple training epochs using a training data set. In some examples, the training of the silent speech machine learning model is performed when the wearable silent speech device is not in use, for example when the device is not being worn or is charging.

In some examples, performing training of the silent speech machine learning model involves retrieving a training dataset from data storage. In some examples, the training dataset may include data previously recorded by the wearable silent speech device from the user of the wearable silent speech device, data previously recorded using a wearable silent speech device from individuals other than the user of the wearable silent speech device, and simulated data representative of a user speaking while using a wearable silent speech device. In some examples, performing training of the silent speech machine learning model involves performing gradient descent using the training dataset, regression analysis using the training dataset, stochastic gradient descent using the training dataset, or momentum training using the training dataset. The training may be performed as described herein. For example, the training dataset may include ground truth data and associated recordings which are used in training. In some examples, the ground truth data may include known words or phrases spoken by the user. The ground truth data may be compared to associated signals or recordings within the training dataset to determine the adjustments to the machine learning model. In some examples, the training of the machine learning model may be performed until a loss function of the silent speech machine learning model is below a threshold value.

In some examples, speech signals recorded using the sensors of the wearable silent speech device may be stored in data storage of the wearable silent speech device prior to use in adjusting machine learning models or analysis. In some examples, speech signals recorded using the sensors of the wearable silent speech device may be stored externally from the silent speech device, for example in an external device connected to the wearable silent speech device or in cloud storage accessible by the wearable silent speech device. In some examples, recorded speech signals may be stored in volatile storage for immediate use or near-immediate use in adjusting the silent speech machine learning model, such as in calibration described herein. For example, a specified length of signals may be recorded, such as the last 3 seconds, last 4 seconds, last 5 seconds, last 5-10 seconds, last 10-15 seconds or last 15-30 seconds of speech signals may be stored in volatile storage and used in a calibration of the silent speech machine learning model. In some examples, speech signals stored in volatile storage are overwritten with newly recorded signals after being used in adjusting the silent speech machine learning model. In some examples, speech signals stored in volatile storage are overwritten continuously.

In some examples, recorded speech signals may be stored in nonvolatile storage for later use. For example, speech signals may be stored in nonvolatile storage for later use in training the silent speech machine learning model. In some examples, speech signals are maintained in nonvolatile storage until they are used in training the silent speech machine learning model and are overwritten after use in training. In some examples, speech signals are maintained in nonvolatile storage permanently. In some examples, speech signals stored in nonvolatile storage for later use are analyzed to determine whether they are suitable for storage. In some examples, the nonvolatile storage is external to the wearable silent speech device, for example the nonvolatile storage may be maintained in an external device connected to the wearable silent speech device or may be cloud storage accessible by the wearable silent speech device.

In some examples, speech signals recorded by a wearable silent speech device may be analyzed to determine if they are suitable for storage including volatile and non-volatile storage. In some examples, it may be determined that speech signals are suitable for storage when it is determined that the signals are associated with: speech not directed at the wearable silent speech device; speech directed at the wearable silent speech device; the user speaking one or more known words or phrases; the user is speaking out loud; the user is speaking silently; data from two or more sensors of the wearable silent speech device is received; data is received from specific sensors or combinations of sensors of the wearable silent speech device such as EMG sensors, a microphone, IMU sensors, EMG sensors and a microphone, EMG sensors and IMU sensors, IMU sensors and a microphone, EMG sensors, IMU sensors and a microphone, among other sensors or combinations of sensors as discussed herein; when a quality metric of the data is above a threshold level; and/or when a level of noise in the recorded signals is below a threshold level.

In some examples, signals may be processed before being stored. For example, signals may be processed by one or more machine learning models, tagged, aligned, cropped, normalized, phonetically balanced, or may undergo other processing as described herein.

In some examples, it is determined whether a machine learning model is to be adjusted before an adjustment, such as calibration or training, of the machine learning is performed.

In some examples, it is determined that an adjustment of the silent speech model is to be performed when speech signals are recorded from two or more sensors of the wearable silent speech device. For example, if the silent speech device records signals from a microphone and an EMG sensor, an EMG sensor and an IMU sensor, a microphone and an IMU sensor, or any other combination of two or more sensors of a silent speech device, as discussed herein, it may be determined that there is sufficient data for adjusting the silent speech model and the silent speech model may be adjusted.

In some examples, it is determined that an adjustment of the silent speech model is to be performed when the user speaks one or more known words or phrases. For example, the signals recorded by the wearable silent speech device may be analyzed to determine if the user has spoken one or more known words of phrases. For example, the signals recorded by the wearable silent speech device may be analyzed by performing template matching of the recorded signals to one or more known signals, or by analyzing the recorded signals using one or more machine learning models to determine if the user has spoken a known word or phrase, among other techniques. Examples of known words or phrases include commands to the device and passwords or passphrases for the device. For example, commands to the device may include “change to silent mode”, “answer call”, “increase volume”, among other commands. In some examples, the signals associated with the known command may be compared to the recorded speech signals when adjusting the silent speech model.

In some examples, it is determined that an adjustment of the silent speech machine learning model is to be performed when the device is being used for the first time or when the device is being turned on, as a part of an onboarding calibration. In some examples, for an onboarding calibration, the user may be prompted with one or more words or phrases to speak, which are recorded and used to calibrate the silent speech machine learning model, as described in herein. In some examples, the user may be asked to choose a password or passphrase which the user may be requested to speak each time the device is used. The password or passphrase may be used as a known signal for adjusting the silent speech machine learning model, for example calibrating the machine learning model.

In some examples, it is determined that an adjustment of the silent speech model is to be performed when a performance metric of the wearable silent speech device exceeds a threshold performance metric level. In some examples, it is determined that an adjustment is to be performed when the performance metric is greater than a threshold level, for example it is determined that an adjustment is to be performed in response to a metric indicative of the number of transcription errors being greater than a threshold level. In some examples, it is determined that an adjustment is to be performed in response to a performance metric being below a threshold level, for example it is determined that an adjustment is to be performed in response to a metric indicative of transcription accuracy dropping below a threshold level. In some examples the performance metric may be indicative of the quality of the output of a machine learning model which analyzes the signals recorded by the silent speech device. In some examples, the output quality may be determined by comparing an output of the machine learning model to a known output value. For example, if a machine learning model is configured to determine words or phrases associated with the recorded signals, and predicts “the gray dog”, however the known output value is “today's long” the quality value may be low, and it is determined that the silent speech machine learning model is to be adjusted. In some examples, the performance metric may be based on a confidence value associated with the output of the machine learning model. In some examples, the performance metric may be based on an error between the output of the silent speech machine learning model and signals recorded by the wearable silent speech device, for example the difference between an audio reconstruction output by the silent speech machine learning model and audio recorded by a sensor of the wearable silent speech device. In some examples, if the error between the output of the silent speech machine learning model and signals recorded by the wearable silent speech device is above a threshold difference it is determined that adjustment is to be performed. In some examples, the performance metric may be based on a word error rate may be determined from a transcript output from the silent speech machine learning model based on a comparison to a transcript output by a second machine learning model such as an automatic speech recognition model. In some examples, it is determined that adjustment is to be performed if the word error rate is above a threshold level. In some examples, the performance metric is based on the number of times a user corrects the output of the silent speech machine learning model. For example, if the user corrects the output more than a threshold number of times, it is determined that an adjustment is to be performed.

In some examples, it is determined that an adjustment of the silent speech model is to be performed based on a user input to the wearable silent speech device. For example, the user may select a button of the silent speech device, the user may provide an input to a connected external device, or may speak one or more known phrases which trigger calibration, such as “begin calibration” or similar. The wearable silent speech device may recognize user inputs to adjust the model and may trigger the model adjustment accordingly.

In some examples, it is determined that an adjustment of the silent speech model is to be performed when the user performs a specific action with the wearable silent speech device. For example, if the user turns on the device, turns off the device, restarts the device, starts charging the device, or stops charging the device, it may be determined that the silent speech machine learning model is to be adjusted.

In some examples it is determined that an adjustment of the silent speech machine learning model is to be performed when a quality metric of the recorded speech signals is above or below a threshold. For example, it may be determined that an adjustment is to be performed in response to determining a noise level in the recorded speech signals is below a threshold noise level.

In some examples, it is determined that an adjustment of the silent speech machine learning model is to be performed when the user is speaking to the device. In some examples, it is determined that an adjustment of the silent speech machine learning model is to be performed when the user is not speaking to the device.

In some examples, it is determined that an adjustment is to be performed at set time periods. For example, training or calibration is to be performed every day, every other day, every 3 days, every 5 days, weekly, every other week, every 3 weeks, monthly, or every other month, among other time periods. In some examples, it is determined that an adjustment is to be performed when a time period has passed since the last adjustment. Calibration may be performed on a shorter time period than training. For example, a calibration adjustment may be performed in response to determining that 1 minute has passed since the last calibration, 2 minutes have passed, 5 minutes have passed, 10 minutes have passed, 30 minutes have passed, 1 hour has passed, 2 hour have passed, among other time periods. For example, a training adjustment may be performed in response to determining that 1 day has passed since the last training, 2 days have passed, 3 days have passed, 4 days have passed, 5 days have passed, 1 week has passed, 2 weeks have passed, 3 weeks have passed, one month has passed, two months have passed, among other time periods. In some examples, it is determined that training is to be performed when the wearable silent speech device has been used for a set period without training. For example, if the wearable silent speech device has been used for 10 hours, 20 hours, 50 hours, or 100 hours, 10-50 hours, or 50-100 hours, without training. In some examples, it may be determined that an adjustment is to be performed when a prompt is received requesting that an adjustment be performed. For example, a user of the device may prompt an adjustment via an input to the wearable silent speech device, by speaking a known word or phrase or via an external device to perform an adjustment. In some examples, an adjustment may be prompted by an external source, for example an administrator of a network to which the wearable silent speech device is connected. In some examples, it is determined that an adjustment is to be performed when one or more actions are performed with the wearable silent speech device. For example, it may be determined that an adjustment is to be performed when the wearable silent speech device is charging, when the wearable silent speech device is turned off, when the wearable silent speech device is turned on, or when the wearable silent speech device has not been used for a set period of time but is still turned on. In some examples, it may be determined that an adjustment is to be performed based on a quality metric of the silent speech machine learning model output. For example, when the average quality metric of the silent speech machine learning model output falls below a threshold level, it is determined that an adjustment is to be performed.

In some examples, calibration of the silent speech machine learning model may occur simultaneously with other processes during the use of the wearable silent speech device. In some examples, training of the silent speech machine learning model may occur simultaneously with other processes during the use of the wearable silent speech device. In some examples, the outputs of the silent speech machine learning model and speech machine learning model may be used to perform additional functions such as communication, transcription, and control, among other functions.

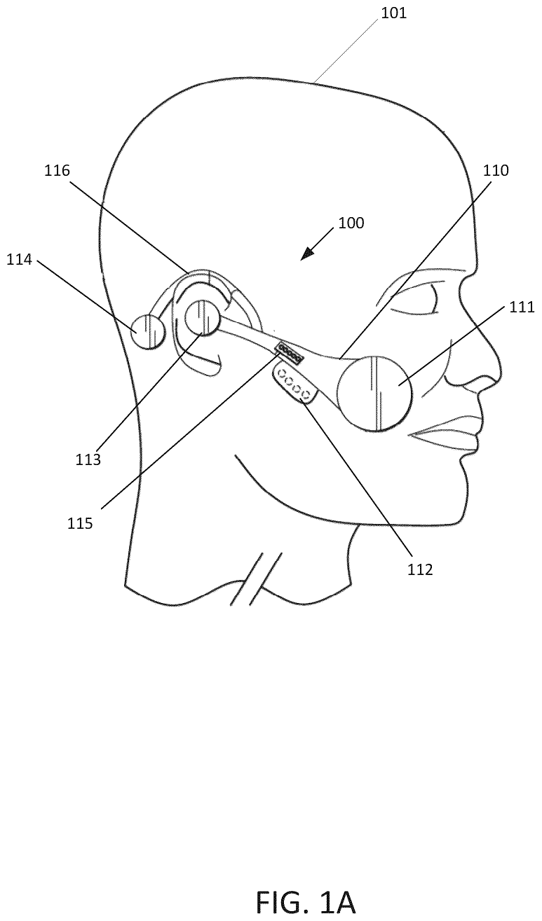

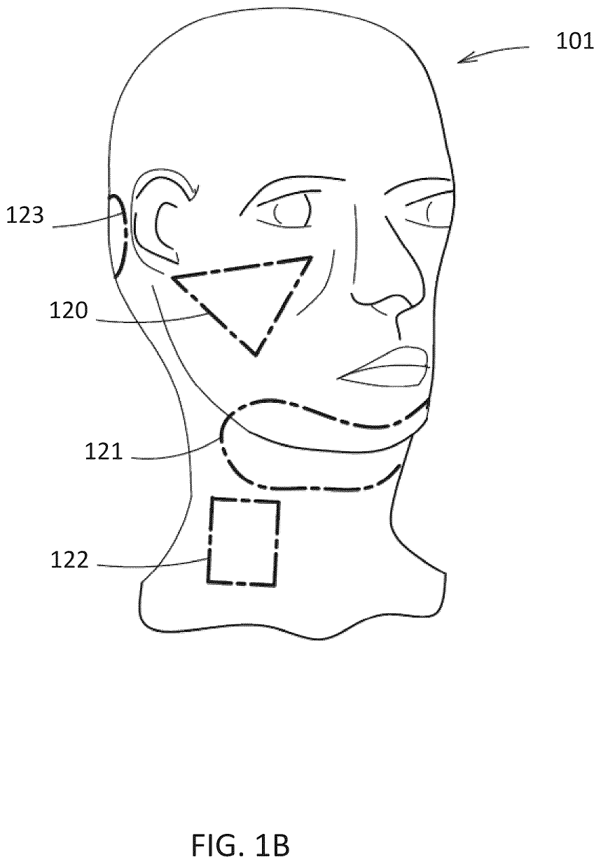

FIG. 1A depicts a view of a user 101 wearing an embodiment of a wearable device 100, in accordance with some embodiments of the technology described herein. The wearable device may comprise an car hook 116 configured to fit around the top of a user's car. The car hook 116 may support a sensor arm 110 of the wearable device 100 and a reference electrode 114 of the device. The car hook may be adjustable to conform to the anatomy of a user. The wearable device 100 may additionally include one or more inputs 115, accessible to the user 101 while the wearable device 100 is being worn. The one or more inputs 115 may include buttons, switches, sliders, and/or capacitive sensors, among other inputs. In some examples, a user may make an input to such input sensors, and in response the wearable device may change states or modes. The input sensors may also be used to turn a wearable device fully off or on.

The wearable device may comprise a sensor arm 110, supported by the ear hook 116. The sensor arm 110 may contain one or more sensors for recording speech signals from the user 101. The one or more sensors supported by the sensor arm may include EMG electrodes 111 configured to detect EMG signals associated with speech of the user. The EMG electrodes may be configured as an electrode array or may be configured as one or more electrode arrays supported by the sensor arm 110 of the wearable device 100.

In some examples, the wearable device may be connected to an external device which may provide inputs to the wearable device. For example, the wearable device may be connected to a smartphone, tablet, computer, laptop computer, desktop computer, a presentation device, smart watch, smart ring, another smart wearable device, among other devices. The external devices may have one or more input sensors which allow the user 101 to provide an input to the external device. The external device may then transmit a signal indicative of the input to the wearable device. The wearable device may then perform one or more actions in response to the signals indicative of the input to the external device.

In some examples, the EMG electrodes 111 may be configured as a differential amplifier, wherein the electrical signals represent a difference between a first voltage measured by a first subset of electrodes of the plurality of electrodes and a second voltage measured by a second subset of electrodes of the plurality of electrodes. Circuitry for the differential amplifier may be contained within the wearable device 100.

The sensor arm may support additional sensors 112. The additional sensors 112 may include a microphone for recording voiced or whispered speech, and an accelerometer or IMU for recording vibrations associated with speech such as glottal vibrations produced during voiced speech. In some examples the IMU may additionally or alternatively be used to measure facial movements. In some examples the wearable device includes multiple IMUs including at least one IMU configured to measure vibrations associated with speech and at least one IMU configured to measure facial movements. In some examples IMUs may be filtered at different frequencies, depending on whether they are measuring speech vibrations or facial motion. For example, IMU filtering at a lower frequency, for example 5-50 Hz, may measure facial motion related to speech and IMU filtering at a higher frequency, for example 100+ Hz, may measure vibrations associated with speech. The additional sensors 112 may include sensors configured to measure a position of a user's tongue, blood flow of the user, muscle strain of the user, muscle frequencies of the user, temperatures of the user, and magnetic fields of the user, among other signals. The additional sensors 112 may include photoplethysogram sensors, photodiodes, optical sensors, laser doppler imaging, mechanomyography sensors, sonomyography sensors, ultrasound sensors, infrared sensors, functional near-infrared spectroscopy (fNIRS) sensors, capacitive sensors, electroglottography sensors, electroencephalogram (EEG) sensors, and magnetoencephalography (MEG) sensors, among other sensors.

In some examples, the wearable device may comprise one or more sensors which detect whether the wearable device 100 is properly positioned on the user. In some examples, the wearable device may include an optical sensor which detects whether the device is properly positioned on the user, for example the optical sensor may determine placement within the car of the user, or on the check of the user, among other locations. In some examples, the wearable device may determine the impedance levels of the EMG electrodes 111 and may determine if the wearable device 100 is properly positioned on the user based on the determined impedance levels. The EMG electrodes 111 may exhibit low impedance if the sensors are properly positioned on the user and high impedance if the sensors are not properly positioned. The wearable device 100 may determine the impedance level of any suitable number of EMG electrodes 111, for example, a single electrode may be checked, 2 electrodes may be checked, 3 electrodes may be checked, any subset of the electrodes may be checked, or all electrodes may be checked. The wearable device 100 may determine it is properly positioned when a threshold number of electrodes are determined to have low impedance. The threshold number of electrodes may be any suitable number of electrodes and may be based on the number of electrodes checked for impedance levels. For example, the threshold number of electrodes may be one electrode, 2 electrodes, 3 electrodes, or any subset of electrodes determined to have low impedance. In some examples, the wearable device may not record, analyze and/or process signals from sensors of the wearable device until it is determined the wearable device is properly positioned on the user 101.

In some examples, the wearable device 100 may determine whether the electrodes are properly placed by applying a known electrical current to the EMG electrodes 111. If the device is not properly placed on the user, one or more of the EMG electrodes may not be contacting the skin of the user. When the known current is applied to EMG electrodes which are not contacting the skin of the user, a voltage may be developed on the inputs of the EMG electrodes, which modulates the input values and is measurable at the EMG electrode outputs. Based on measured voltage, it may be determined the impedance of the of the signal from EMG electrode inputs is high. In some examples, analog to digital conversion of the voltage may be performed on the signal measured at the EMG electrode outputs to determine the impedance. The wearable device determines based on the measured voltage and/or determined impedance, the wearable device 100 is not properly placed on the user.

In some examples, when the known current is applied to EMG electrodes which are properly placed on the user (contacting the skin of the user), the current may flow through the EMG electrodes to the skin of the user and back to the device via another connection to the body, for example the reference electrode 114. This path for the known current is in parallel with the EMG electrode inputs, however has a much lower impedance than the EMG electrode inputs. The wearable device may determine the impedance based on the measured signals. The wearable device may therefore, based on the determined lower impedance, recognize the EMG electrodes are properly placed on the user.

In some examples, the known current is a DC current, which generates a constant voltage. The voltage may vary based on the impedance of the current path, which may be recognized by the wearable device 100. In some examples, the known current is an AC current, which is applied at a known frequency. In some examples, the known AC current may be characterized by its peak current. The known AC current creates a voltage which may be recognized by the wearable device 100 through extraction from sensor data using frequency decomposition techniques such as Fourier or quadrature methods. The wearable device 100 may perform EMG electrode and impedance measurement through the same sensor, when a known AC current is used because of the bandwidth of the signal having multiple current levels which can be used.

In some examples, the car hook 116 may additionally support one or more reference electrodes 114. The reference electrode 114 may be placed are located on a side of the car hook 116, facing the user 101. In some examples reference electrode 114 may be configured to bias the body of the user such that the body is an optimal range for sensors of the system including sensors 112 and EMG electrodes 111. In some examples, the reference electrode 114 is configured to statically bias the body of the user. In some examples, the reference electrode 114 is configured to dynamically bias the body of the user.

The wearable device 100 may include a speaker 113 positioned at an end of the sensor arm. The speaker 113 is positioned at the end of the sensor arm 110 closest the user's car. The speaker 113 may be inserted into the user's car to play sounds, may perform bone conducting to play sounds to the user, or may play sounds aloud adjacent to the user's car. The speaker 113 may be used to play outputs of silent speech processing or communication signals as discussed herein. In addition, the speaker 113 may be used to play one or more outputs from a connected external device, or the wearable device, such as music, audio associated with video or other audio output signals.

The wearable device 100 may include other components which are not pictured. These components may include a battery, a charging port, a data transfer port, among other components.

Wearable device 100 is an example of a wearable device which may be used in some embodiments of the technology described herein. Additional examples of wearable devices which may be used in some embodiments of the technology described herein include are described in U.S. patent application Ser. No. 18/338,827, titled “WEARABLE SILENT SPEECH DEVICE, SYSTEMS, AND METHODS”, filed Jun. 21, 2023, and with attorney docket number W1123.70003US00, the entirety of which is incorporated by reference herein.

FIG. 1B is an illustration of wearable device target zone(s) associated with a wearable speech input device such as wearable device 100 (FIG. 1A), in accordance with some embodiments of the technology described herein. The target zones may include one or more areas on or near the user's body part, in which sensor(s) can be placed to measure speech muscle activation patterns while the user is speaking (silently or with voice) or preparing to speak. For example, the speech muscle activation patterns at various target zones may include facial muscle movement, neck muscle movement, chin muscle movement, or a combination thereof associated with the user speaking. In some examples, the sensors may be placed at or near a target zone at which the sensors may be configured to measure the blood flow that occurs as a result of the speech muscle activation associated with the user speaking. Thus, the wearable device 100 may be configured to have its sensors positioned to contact one or more target zones, such as the face and neck of the user.

With further reference to FIG. 1B, various target zones are shown. In some embodiments, a first target zone 120 may be on the check of the user 101. This first target zone 120 may be used to record electrical signals associated with muscles in the face and lips of the user, including the zygomaticus of the user, the masseter of the user, the buccinator of the user, the risorius of the user, the platysma of the user, the orbicularis oris of the user, the depressor anguli oris of the user, the depressor labii, the mentalis, and the depressor septi of the user.

In some embodiments, various sensors may be positioned at the first target zone 120. For example, electrodes (e.g., 111 in FIG. 1A) supported by the wearable device 100 (e.g., via a sensor arm 110) may be positioned to contact the first target zone 120 of the user. In some embodiments, sensors configured to measure the position and activity of the user's tongue may be supported at the first target zone 120 by the sensor arm. In some embodiments, accelerometers configured to measure movement of the user's face may be placed at the first target zone 120.

In some embodiments, a second target zone 121 is shown along the jawline of the user. The second target zone 121 may include portions of the user's face above and under the chin of the user. The second target zone 121 may include portions of the user's face under the jawline of the user. The second target zone 121 may be used to measure electrical signals associated with muscles in the face, lips jaw and neck of the user, including the depressor labii inferioris of the user, the depressor anguli oris of the user, the mentalis of the user, the orbicularis oris of the user, the depressor septi of the user, the mentalis of the user, the platysma of the user and/or the risorius of the user. Various sensors may be placed at the second target zone 121. For example, electrodes (e.g., 111 in FIG. 1A) supported by the wearable device 100 (e.g., via a sensor arm 110) may be positioned to contact the second target zone 121. Additional sensors, e.g., accelerometers, may be supported by the wearable device and positioned at the second target zone 121 to measure the movement of the user's jaw. Additional sensors may also include sensors configured to detect the position and activity of the user's tongue.

In some embodiments, a third target zone 122 is shown at the neck of the user. The third target zone 122 may be used to measure electrical signals associated with muscles in the neck of the user, e.g., the sternal head of sternocleidomastoideof the user, or the clavicular head of sternocleidomastoideous sensors may be positioned at the third target zone 122. For example, accelerometers may be supported at the third target zone to measure vibrations and movement generated by the user's glottis during speech, as well as other vibrations and motion at the neck of user 101 produced during speech.

In some embodiments, a reference zone 123 may be located behind the ear of the user at the mastoid of the user. In some embodiments, reference electrodes (e.g., 114 in FIG. 1A) may be positioned to contact the reference zone 123 to supply a reference voltage to the face of the user, as discussed herein. Reference zone 123 may also include portions of the user's head behind and above the car of the user.

With reference to FIGS. 1A and 1B, as discussed with reference to multiple target zones for measuring the user's speech muscle activation patterns associated with the user speaking, the wearable device 100 may include various mechanisms to adjust the positions of sensors for accommodating one or more target zones. For example, the sensor arm (e.g., 110) of the wearable device 100 may be adjustable along the axis of the sensor arm to enable the electrodes (e.g., 111 in FIG. 1A) on the sensor arm to align with a target zone. In some embodiments, one or more parts of the wearable device 100 may be moveable laterally, for example, to enable the sensor(s) thereon to be closer or further away from the user's body part (e.g., face or neck). In some embodiments, the wearable device 100 may include multiple sensor arms wearable on both sides of the face to enable multiple sets of sensors on either or both sides of the face or neck. It is appreciated that other suitable configurations may be possible to enable any sensors to be suitably positioned in respective target zones.

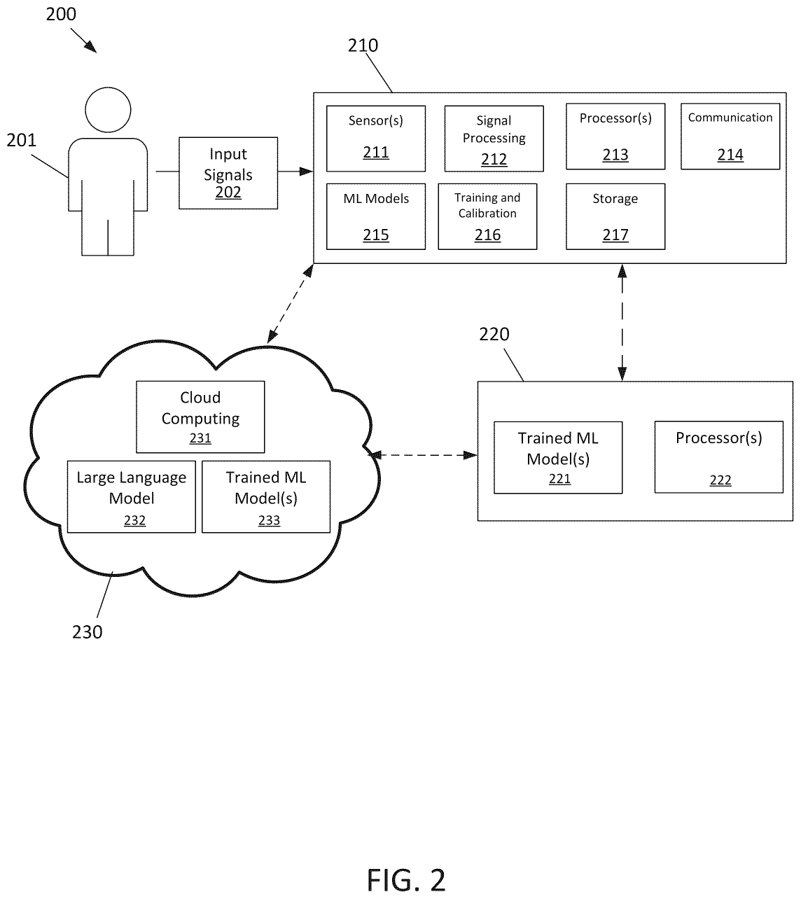

FIG. 2 is a block diagram of a silent speech system 200, in accordance with some embodiments of the technology described herein. The silent speech system 200 may include a wearable device 210, an external device 220 and a server 230.

The wearable device may be configured to record input signals 202 of a user 201. The signals 202 may include signals associated with silent speech or voiced speech, as described herein.

Specific modules are shown within the external device 220 and server 230, however these modules may be located within any of the wearable device 210, external device 220 and server 230. In some examples, the external device 220 may contain the modules of the server 230 and the wearable device 210 will communicate directly with the external device 220. In some examples, the server 230 may contain the modules on the external device 220 and the wearable device 210 will communicate directly with the server 230. In some examples, the wearable device 210 may contain the modules of both the external device 220 and the server 230 and therefore the wearable device 210 will not communicate with the external device 220 or the server 230 to determine one or more words or phrases from the signals 202 recorded by the wearable device. In some examples some modules of the server 230 and external device 220 may be included in the server 230, external device 220 and/or the wearable device 210. Any combination of modules of the server 230 or external device 220 may be contained within the server 230, the external device 220 and/or the wearable device 210.

The wearable device 210 may include one or more sensors 211 which are used to record signals 202 form a user. The sensors 211 may include EMG electrodes for recording muscle activity associated with speech, a microphone for recording voiced and/or whispered speech, an accelerometer or IMU for recording vibrations associated with speech and other sensors for recording signals associated with speech. These other sensors may measure a position of a user's tongue, blood flow of the user, muscle strain of the user, muscle frequencies of the user, temperatures of the user, and magnetic fields of the user, among other signals, and may include: photoplethysogram sensors, photodiodes, optical sensors, laser doppler imaging, mechanomyography sensors, sonomyography sensors, ultrasound sensors, infrared sensors, Functional near-infrared spectroscopy (fNIRS) sensors, capacitive sensors, electroglottography sensors, electroencephalogram (EEG) sensors, and Magnetoencephalography (MEG) sensors, among other sensors.

The sensors 211 may be supported by the wearable device to record signals 202 associated with speech, either silent or voiced, at or near the head, face and/or neck of the user 201. Once recorded, the signals may be sent to a signal processing module 212 of the wearable device 210. The signal processing module 212 may perform one or more operations on the signals including filtering, thresholding, and analog to digital conversion, among other operations.

The signal processing module 212 may then pass the signals to one or more processors 213 of the wearable device 210. In some examples, the processors 213 may process the signals to determine if the user is speaking silently or voiced and may determine one or more words or phrases from the signals and compare these words or phrases to known commands to determine an action the user wishes to perform. In some examples, additionally the processors may process the signals to determine if the user is preparing to speak. The processors may perform any additional functions of the wearable device, as described herein.

In some examples, the processors 213 may perform additional processing on the signals including preprocessing, and digital processing. In addition, the processors may utilize one or more machine learning models 215 stored within the wearable device 210 to process the signals. The machine learning models 215 may be used to perform operations including feature extraction, and downsampling, as well as other processes for recognizing one or more words or phrases from signals 202. In some examples, the processes for recognizing words or phrases using the ML models 215 may involve determining words spoken by the user from signals 202 using automatic speech recognition (ASR). In some examples, the processes for recognizing words or phrases using the ML models 215 may involve determining signals which are associated with words or phrases spoken by the user, however, are not direct representations of the words or phrases. For example, the ML models 215 may output an audio signal associated with words or phrases silently spoken by the user 201. In some examples, the ML models 215 outputs may include encoded representations of the words or phrases spoken by the user.

The wearable device 210 also includes model training and calibration module 216. The model training and calibration module may be used to adjust the ML models 215. In some examples, the ML models 215 may be adjusted based on signals 202 recorded from user 201. In some examples, the ML 215 models may be adjusted on data stored in data storage 217. In some examples, the ML models 215 may be adjusted based on a combination of signals 202 recorded from the user 201 and data stored in data storage 217.

After processing, signals may be sent to communication module 214, which may transmit the signals to one or more external devices or systems. The communication module 214 may perform one or more operations on the processed signals to prepare the signals for transmission to one or more external devices or systems. The signals may be transmitted using one or more modalities, including but not limited to wired connection, Bluetooth, Wi-Fi, cellular network, Ant, Ant+, NFMI and SRW, among other modalities. The signals may be communicated to an external processing device and/or to a server for further processing and/or actions.

The one or more external devices or systems may be any device suitable for processing silent speech signals including smartphones, tablets, computers, purpose-built processing devices, wearable electronic devices, and cloud computing servers, among others. In some examples, the communication module 214 may transmit speech signals directly to a server 230, which is configured to process the speech signals using one or more processors, as described herein. In some examples, the communication module 214 may transmit speech signals to an external device 220 which processes the signals directly, using one or more processors, as described herein. In some examples, the communication module 214 may transmit speech signals to an external device 220 which, in turn, transmits the signals to server 230 which is configured to process the speech signals. In some examples, the communication module 214 may transmit speech signals to an external device 220 which is configured to partially process the speech signals and transmit the processed speech signals to a server 230 which is configured to complete the processing of the speech signals. The wearable device 210 may receive one or more signals from the external device or the cloud computing system in response to any transmitted signals.

The external device 220 may contain one or more trained ML models 221 and processors 222. The processors may be configured to recognize one or more words or phrases from the signals received from wearable device 210 using the trained ML models 221. In some examples, the processors may execute one or more actions on the external device based on the determined words or phrases. For example, applications and functions of the wearable device may be controlled, text inputs may be provided to the external device and communication may be supported by the external device using the signals received from the wearable device, among other actions.

The external device 220 may communicate with server 230 to perform one or more actions. The external device 220, and server 230 may be connected via a common network. The server may include cloud computing components 231 to facilitate the connection and communication and a large language model (LLM) 232. The LLM 232 may be used to process words or phrases identified from the speech signals and to determine an action to be performed based on the words or phrases. The server also includes trained ML models 233, which may recognize one or more words or phrases from signals 202 recorded by the wearable device 210, or from processed signals received from the wearable device 210 or external device 220.

The ML models 215 of the wearable device, 221 of the external device, and 233 of the server 230, may be structured in any suitable way, such as those described in in U.S. patent application Ser. No. 18/338,827, titled “WEARABLE SILENT SPEECH DEVICE, SYSTEMS, AND METHODS”, filed Jun. 21, 2023, and with attorney docket number W1123.70003US00, the entirety of which is incorporated by reference herein.

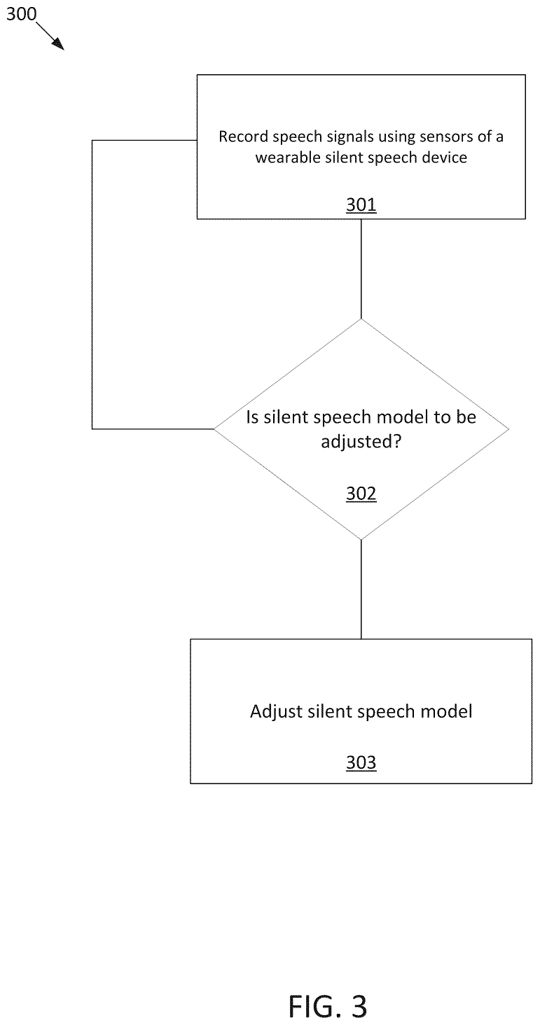

FIG. 3 is an example process flow for adjusting a machine learning model, according to some embodiments of the technology described herein. Process flow 300 begins at step 301, where speech signals are recorded using sensors of a silent speech device. In some examples, the speech signals may be recorded as discussed with relation to FIG. 1A-B. In some examples, the speech signals may be recorded using at least two sensors of the silent speech device, for example, a microphone and an EMG sensor, an EMG sensor and an IMU sensor, a microphone and an IMU sensor, or any other combination of sensors of a silent speech device, as discussed herein. In some examples, the sensor may be recorded using two sensors of the silent speech device, three sensors of the silent speech device, four sensors of the silent speech device, five sensors of the silent speech device, up to ten sensors of the silent speech device or greater than ten sensors of the silent speech device. In some examples, speech signals may be recorded by sensors of the silent speech device and speech signals may additionally be recorded by sensors of an external device. For example, a microphone of an external device may record voice signals of the user, or a camera of an external device may record video of a user.

In some examples, recorded signals may be stored as described herein, for example in volatile or nonvolatile storage. In some examples, recorded speech signals may be maintained in volatile or nonvolatile storage. In such examples, the storage may be located within the wearable silent speech device, within an external device or within a server. In examples where the volatile or nonvolatile storage is located in an external device, the recorded signals may be transmitted to the external device from the wearable silent speech device. In examples where the volatile or nonvolatile storage are maintained in a server, the recorded speech signals may be transmitted to the server directly from the wearable silent speech device or indirectly through an external device.

At step 302, it is determined whether the silent speech model is to be adjusted. If it is determined that the silent speech model is to be adjusted, the process proceeds to step 303. If it is determined that the silent speech model is not to be adjusted, the process returns to step 301.

In some examples, it is determined that the silent speech model is to be adjusted when speech signals are recorded from two or more sensors of the wearable silent speech device, as described herein. In some examples, data from the two or more sensors is compared when adjusting the silent speech model in step 303.

In some examples, it is determined that the silent speech model is to be adjusted when the user speaks one or more known words or phrases, as described herein. In some examples it is determined that the user has spoken one or more known words or phrases by performing template matching of the recorded signals to one or more known signals, or by analyzing the recorded signals using one or more machine learning models to determine if the user has spoken a known word or phrase, among other techniques.

In some examples, it is determined that the silent speech model is to be adjusted when it is determined a performance metric of the wearable silent speech device is below a threshold performance metric level, as described herein.

In some examples, it is determined that the silent speech model is to be adjusted based on a user input to the wearable silent speech device. For example, the user may select a button of the silent speech device, such as those of inputs 115 of FIG. 1A, the user may provide an input to a connected external device such as 220 of FIG. 2, or may speak one or more known phrases which trigger calibration, such as “begin calibration” or similar. The wearable silent speech device may recognize user inputs to adjust the model and may trigger the model adjusting accordingly.

In some examples, it is determined that the silent speech model is to be adjusted when the user performs a specific action with the wearable silent speech device. For example, if the user turns on the device, turns off the device, restarts the device, starts charging the device, stops charging the device, it may be determined that the silent speech machine learning model is to be adjusted.

At step 303, the silent speech machine learning model is adjusted. In some examples, adjusting the machine learning model may involve performing one or more training processes based on the signals recorded by the wearable silent speech device, as described herein. In some examples, adjusting the silent speech machine learning model includes performing calibration of the silent speech machine learning model, as described herein. In some examples, adjusting the silent speech machine learning model includes performing training of the silent speech machine learning model, as described herein.

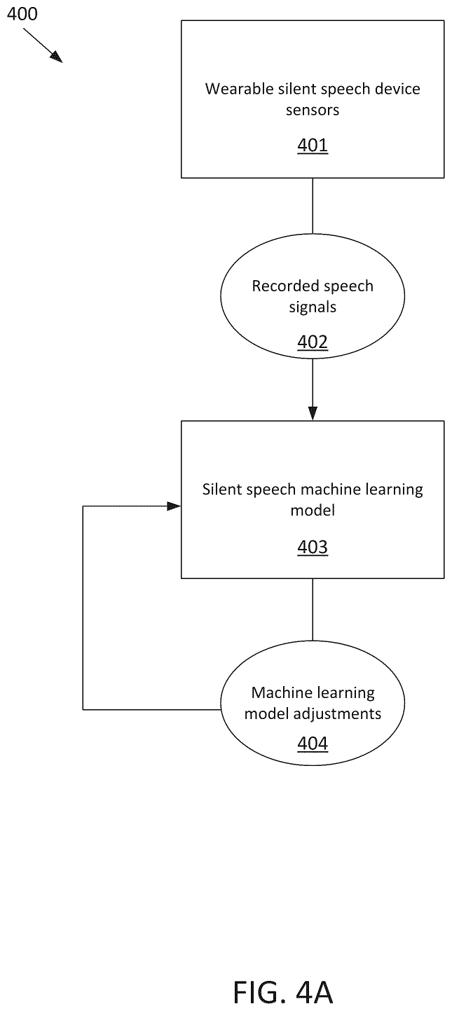

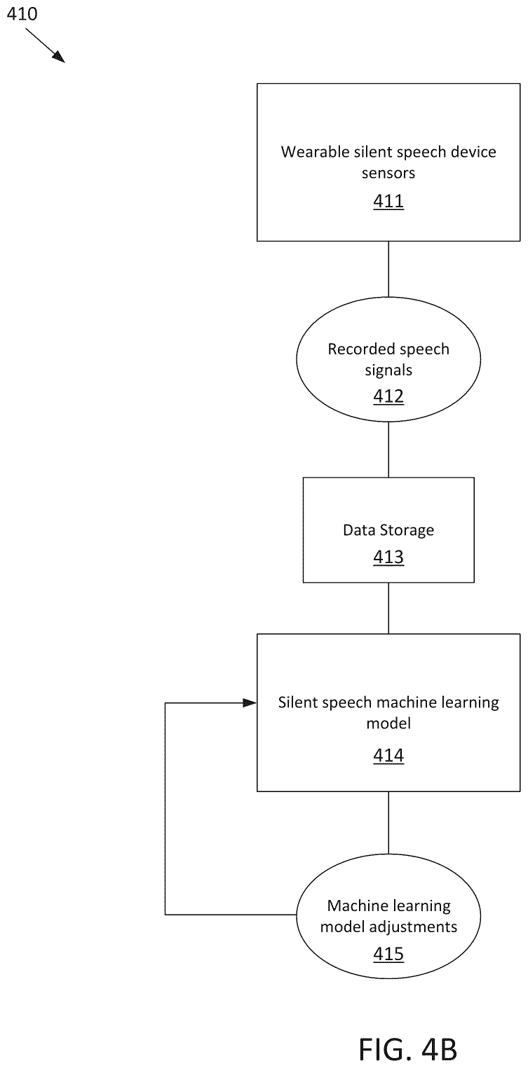

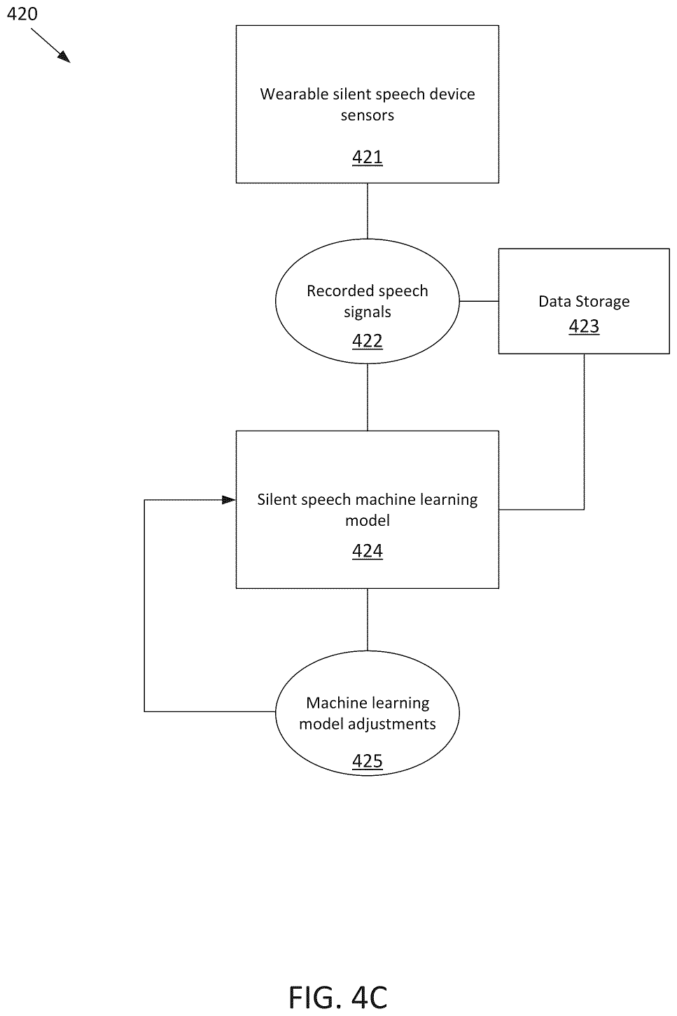

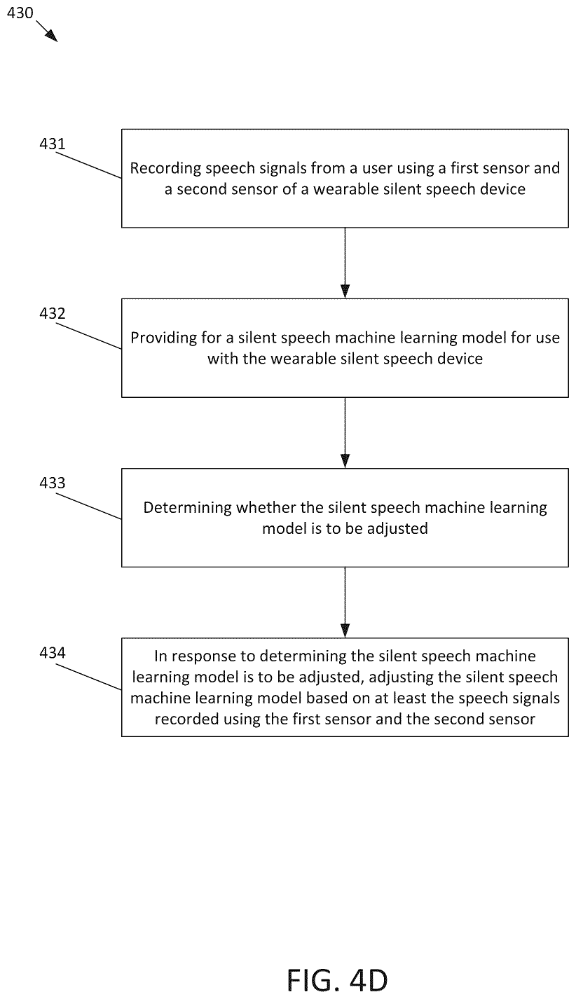

FIG. 4A shows an example data flow for performing calibration of a silent speech machine learning model, according to embodiments of the technology described herein. The process 400 may be performed continually as the wearable silent speech device is being used, in order to adapt the silent speech machine learning model to the current state of the user. As shown, the wearable silent speech device sensors 401 record silent speech signals 402 from a user of the device. The recorded signals 402 are passed to the silent speech machine learning model 403. In some examples, the signals may be processed before being passed to the silent speech machine learning model, as described herein. Processing may include encoding of the signals, data extraction from the signals, filtering of the signals, formatting of the signals, among other processing. In some examples, the signals 402 may be passed to storage, such as volatile storage or a memory buffer before being passed to the silent speech machine learning model 403. The sensors of the wearable silent speech device continuously record signals which may be used to adjust the silent speech machine learning model.

In some examples, the silent speech machine learning model 403 may analyze the recorded speech signals 402. In some examples, the silent speech machine learning model may analyze signals which have been processed. In some examples the silent speech machine learning model may analyze the recorded speech signals 402 and signals which have been processed.

Machine learning model adjustments 404 may be generated based on processing of the signals using the silent speech machine learning model. In some examples, the machine learning model adjustments may be determined by performing one or more training processes, as described herein. In some examples, the machine learning model adjustments may be determined, at least in part by comparing the output of the silent speech machine learning model to ground truth data. In some examples, the machine learning model adjustments may be determined in part by using a second machine learning model in addition to the silent speech machine learning model. In some examples, it is determined whether the silent speech machine learning model is to be adjusted, as discussed herein, and the machine learning model adjustments are determined in response to determining the silent speech machine learning model is to be adjusted.

The machine learning model adjustments 404 may be applied to the silent speech machine learning model 403. The adjusted silent speech machine learning model may then be used in the processing of signals recorded from the wearable silent speech device.

FIG. 4B shows an example data flow 410 for performing training of a silent speech machine learning model according to some embodiments of the technology described herein. As shown, the wearable silent speech device sensors 411 record speech signals 412 from a user with the device. The recorded signals 412 are passed to nonvolatile data storage 413. In some examples, as described herein, it is determined whether the recorded signals are suitable for storage before being passed to data storage 413. In some examples, as described herein, signals are processed before being passed to data storage.

Signals 412 may be maintained in data storage 413 until training of the silent speech machine learning model 414 is performed. When it is determined training of the silent speech machine learning model 414 is to be performed, a data set generated from data stored in data storage 413 is provided to the silent speech machine learning model 414. Model adjustments 415 are determined using the training dataset, as described herein. The silent speech machine learning model adjustments 415 are then applied to the silent speech machine learning model 414. In some examples, the machine learning model adjustments 415 may be determined during training of the silent speech machine learning model by performing multiple training epochs using the data set retrieved from data storage.

FIG. 4C shows an example data flow 420 for performing calibration and training of a silent speech machine learning model, according to some embodiments of the technology described herein. In some examples a wearable silent speech device may perform both calibration and training, as described herein. Recorded speech signals 422 from the wearable silent speech device sensors 421 may be passed directly to the silent speech machine learning model 424 and/or to data storage 423. In some examples, recorded speech signals 422 are analyzed to determine if they are suitable for storage, and/or are processed before storage, as described herein.

In some examples, a silent speech machine learning model 424 may undergo calibration based on speech signals recorded from a wearable silent speech device. In some examples, calibration may be performed in response to determining the silent speech machine learning model is to be adjusted, as described herein. In some examples, calibration may be performed by performing a training iteration of the silent speech machine learning model using data recorded from the sensors, as described herein. In some examples, data used to perform calibration may be passed from volatile data storage, as described herein. In some examples, data used to perform calibration may be passed from nonvolatile data storage, as described herein.

The recorded speech signals 422 may additionally or alternatively be sent to data storage 423 where they are maintained until training of the silent speech machine learning model is performed. The training of the silent speech machine learning model may be performed less frequently than calibration of the silent speech machine learning model. In some examples, performing training of the silent speech machine learning model 424 may involve performing multiple training epochs using the data obtained from data storage 423, as described herein.

Machine learning model adjustments 425 may be determined by performing training of the silent speech machine learning model or by performing calibration of the silent speech machine learning model. Machine learning model adjustments 425 may be applied to silent speech machine learning model 424, as described herein.

FIG. 4D illustrates an example process flow for adjusting a silent speech machine learning model, according to some embodiments of the technology described herein. The process 430 begins with step 431, in which speech signals are recorded from a user using first and second sensors of a wearable silent speech device. The first and second sensors may include any sensors, such as those described with relation to FIG. 1A. Such sensors may include EMG sensors; a microphone, IMU sensors; sensors configured to measure: a position of a user's tongue, blood flow of the user, muscle strain of the user, muscle frequencies of the user, temperatures of the user, and magnetic fields of the user; photoplethysogram sensors; photodiodes; optical sensors; laser doppler imaging; mechanomyography sensors; sonomyography sensors; ultrasound sensors; infrared sensors; functional near-infrared spectroscopy (fNIRS) sensors; capacitive sensors; electroglottography sensors; electroencephalogram (EEG) sensors; and magnetoencephalography (MEG) sensors; among other sensors, as described herein. In some examples, at least one of the first or second sensors is an EMG sensor.

The process then proceeds to step 432, in which a silent speech machine learning model is provided for use with the wearable silent speech device. The silent speech machine learning model may be any suitable model as described herein, including, but not limited to: statistical models, neural networks, and autoregressive machine learning models, flow matching machine learning models, diffusion machine learning models, among other types of machine learning models. The silent speech machine learning model may be provided by the wearable silent speech device, may be provided by a connected device, or may be provided via a network the wearable silent speech device is connected to.

The process then proceeds to step 433, in which it is determined whether the silent speech machine learning model is to be adjusted. In some examples, determining whether the silent speech machine learning model is to be adjusted comprises determining the silent speech machine learning model is to be adjusted: when speech signals are recorded from two or more sensors of the wearable silent speech device, when the user speaks one or more known words or phrases, when the device is being used for the first time, when the device is being turned on, when a performance metric of the wearable silent speech device is below a threshold performance metric level, based on a user input to the wearable silent speech device, when the user performs a specific action with the wearable silent speech device, when a quality metric of the recorded speech signals is above or below a threshold level, when the user is speaking to the device, when the user is not speaking to the device, when the user is speaking out loud, when the user is speaking silently, when the user is whispering, at set time periods, when a time period has passed since the last adjustment, when a prompt is received requesting that an adjustment be performed, in response to determining a noise level in the recorded speech signals is below a threshold noise, among other reasons for triggering the adjustment of the silent speech machine learning model as described herein.

In response to determining the process is to be adjusted, the process proceeds to step 434, in which the silent speech machine learning model is adjusted based on at least the speech signals recorded using the first and second sensors of the wearable silent speech device. Adjusting the silent speech machine learning model may involve any adjustment, calibration or training technique, as described herein.

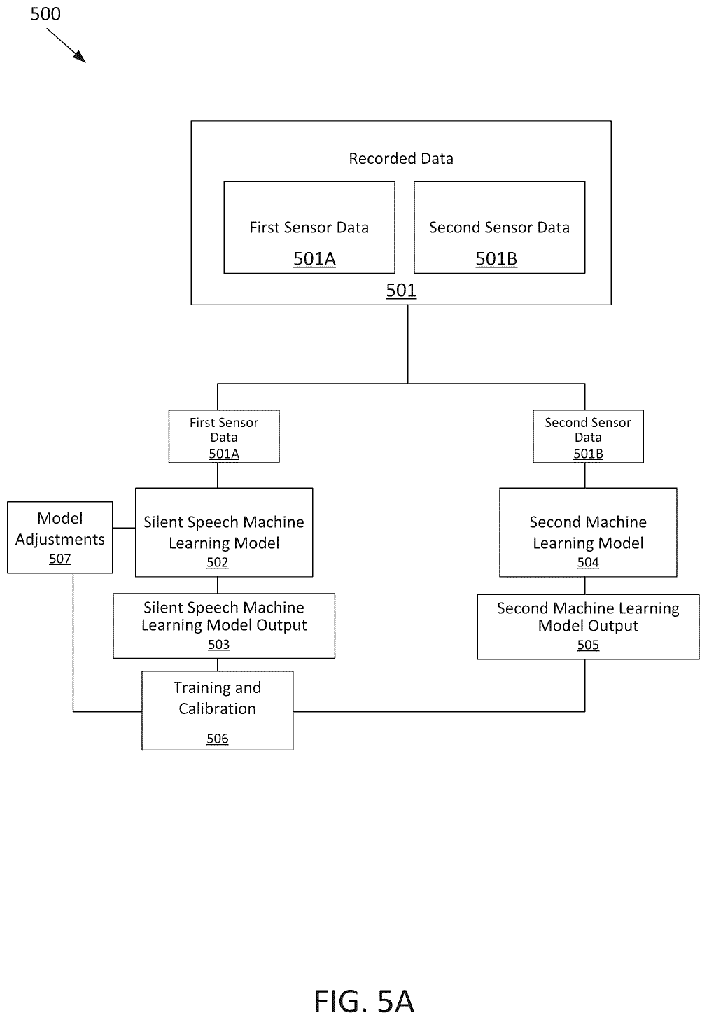

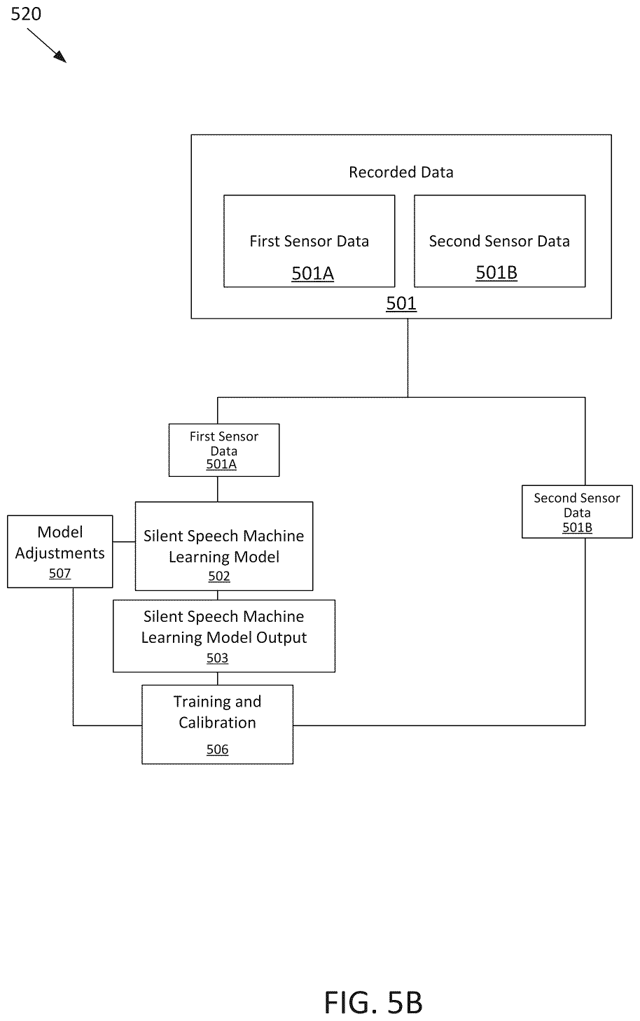

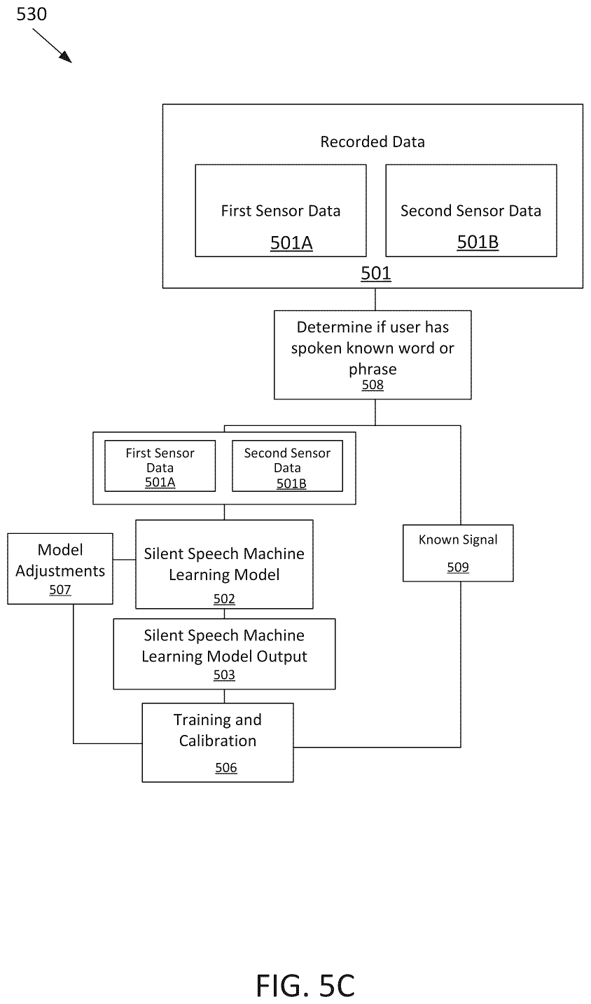

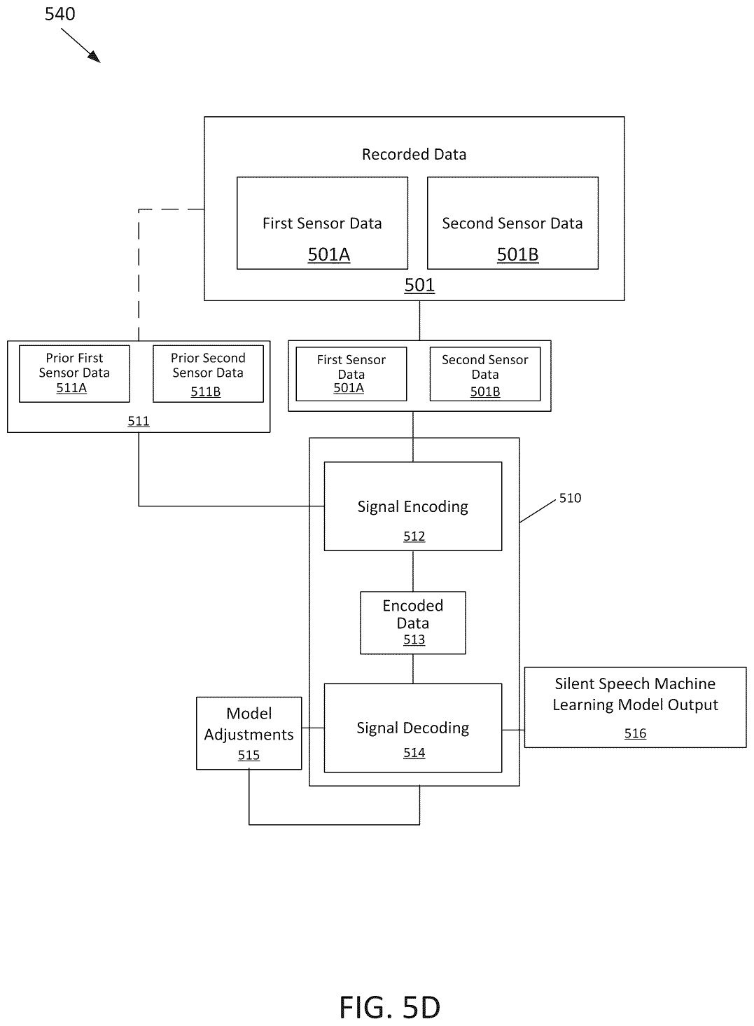

FIGS. 5A-D illustrate example data flows for performing calibration of a silent speech machine learning models, according to some aspects of the technology described herein. In the examples of FIGS. 5A-D, recorded data 501 includes first sensor data 501A recorded from a first sensor of the wearable silent speech device, and second sensor data 501B recorded from a second sensor of the wearable silent speech device. In some examples, recorded data 501 may include data from greater than two sensors of a wearable silent speech device, as discussed herein. In some examples, the data flows of FIGS. 5A-D is performed in response to determining the silent speech machine learning model is to be adjusted. In some examples, the data flows of FIGS. 5A-D are performed in parallel with other functions of a wearable silent speech device, as described herein. In some examples, the data flows of FIGS. 5A-D involves determining whether the silent speech machine learning model is to be adjusted. In some examples, the recorded data 501 is passed to ML models from volatile storage or non-volatile storage, as described herein. In some examples, the recorded data 501 is passed to ML models from the sensors of the wearable silent speech device. In some examples, the recorded data is passed to ML models from a memory buffer. In some examples, the recorded data may undergo processing before being passed to ML models. The first sensor data 501A is passed to silent speech machine learning model 502. In some examples, the first sensor data is recorded from a sensor which records data when a user is speaking silently, such as an EMG sensor, or IMU sensor, among other sensors as discussed herein.

FIG. 5A is an example of performing calibration of the silent speech machine learning model using outputs of the silent speech machine learning model and a second machine learning model, according to some embodiments of the technology described herein. In the data flow 500 of FIG. 5A, the first sensor data 501A is processed by the silent speech machine learning model 502 to determine silent speech machine learning model output 503. In some examples, the silent speech machine learning model output 503 may be a representation of words or phrases spoken by the user. In some examples silent speech machine learning model output 503 may be transcribed words or phrases determined via ASR. In some examples, the silent speech machine learning model output 503 may be a signal indicative of words or phrases spoken by the user, such as an audio signal indicative of words or phrases spoken by the user, or another signal format indicative of words or phrases spoken by the user.

The second sensor data 501B is passed to the second machine learning model 504. In some examples, the second machine learning model may be a model different from the from the silent speech machine learning model 502. In some examples, the second machine learning model 504 is a machine learning model configured for processing signals of specific sensors. For example, the second sensor data 501B may be data recorded by a microphone of a silent speech device and the second ML model may be configured for processing signals recorded by microphones. In some examples, the second ML model 504 may be configured for processing signals of any sensor of a wearable silent speech device, as described herein. The second machine learning model output 505 may be indicative of words or phrases spoken by the user, as described herein. The second machine learning model output 505 may be in the same format as the silent speech machine learning model output 503.

The silent speech machine learning model output 503 and the second machine learning model output 505 are passed to training and calibration module 506. In some examples, the training and calibration module may determine whether the silent speech machine learning model is to be adjusted. Calibration of the silent speech machine learning model may occur simultaneously with other processes during the use of the wearable silent speech device. In some examples, the outputs of the silent speech machine learning model and second machine learning model may be used to perform additional functions such as communication, transcription, and control, among other functions.

In some examples, the training and calibration module 506 may compare the outputs of the silent speech machine learning model 502 and second macing learning model 504 and determine a quality metric based on the difference between the outputs. The quality metric may indicate a quality of the silent speech machine learning model output 503, using the second machine learning model output as a ground truth. A high quality metric value may indicate the silent speech machine learning model 504 is similar to the second machine learning model output 505, and a low quality metric value may indicate the silent speech machine learning model output 505 has many differences from the second machine learning model output 505. In some examples, the training and calibration module 506 may determine the silent speech machine learning model is to be adjusted in response to determining the quality metric value is below a threshold quality metric value.

In some examples the training and calibration module 506 may tag the silent speech machine learning model output 503 with associated data of the second machine learning model output 505, for adjusting of the silent speech machine learning model 502. In some examples, the training and calibration module may tag the silent speech machine learning model output 503 with associated data of the second machine learning model output 505. In some examples, the tagging is performed in response to determining the silent speech machine learning model 502 is to be adjusted. In some examples, the silent speech machine learning model output 503 and second machine learning model output 505 may include representations of one or more words spoken by the user and tagging the silent speech machine learning model output 503 may involve tagging each word representation of the silent speech machine learning model output 503 with the associated word representation of the second machine learning model output 505. In some examples, the silent speech machine learning model output 503 and second machine learning model output 505 do not contain representations of words spoken by the users and tagging the silent speech machine learning model output 503 may involve associating portions of the silent speech machine learning model output 503 with portions of the second learning model output. For example, the tagging may involve temporally aligning the silent speech machine learning model output 503 with the second machine learning model output 505, or aligning the model outputs in any suitable way, for example by frequency. The tagged silent speech machine learning model output may be provided to the silent speech machine learning model to determine model adjustments 507, which are implemented in the silent speech machine learning model 502.

In some examples, the training and calibration module 506 may determine a loss function value based on the silent speech machine learning model output 503 and the second machine learning model output 505. Examples of loss functions which may be used by the training and calibration module 506 include a mean square error, a mean absolute error, a mean bias error, a Huber Loss, a log-cosh loss, a quantile loss, among other loss functions. In some examples, the loss function value is determined by using the second machine learning model output 505 as a ground truth value for calculating the loss function value and the silent speech machine learning model output 503 is used as a prediction value for calculating the loss function value. In some examples, the loss function value is used to perform gradient descent with respect to one or more weights of the silent speech machine learning model 502. One or more weight adjustments may be determined by performing gradient descent based on the loss function value. The weight adjustments may be provided to the silent speech machine learning model 502 as model adjustments 507.

The model adjustments 507 may be provided to the silent speech machine learning model 502. The silent speech machine learning model 502 may be adjusted based on the model adjustments 507. Adjusting the model may involve changing one or more weights of the model, changing one or more features of the model or changing one or more parameters of the model.

In some examples, calibration may be performed continuously as the user is wearing and/or using the wearable silent speech device. In some examples, calibration may be performed at set intervals when the user is wearing or using the wearable silent speech device, such as time intervals, for example every minute, two minutes, 3 minutes, 4 minutes, 5 minutes, 5-10 minutes, or 10-20 minutes, or word intervals, for example every 100 words, 500 words, 1,000 words, 2,500 words, 5,000 words, or 10,000 words.

FIG. 5B illustrates an example data flow 520 for adjusting a silent speech machine learning model using signals recorded by sensors of a wearable silent speech device, according to some embodiments of the technology described herein. As shown, recorded data 501 includes first sensor data 501A recorded from a first sensor of the wearable silent speech device, and second sensor data 501B recorded from a second sensor of the wearable silent speech device. In some examples, recorded data 501 may include data from greater than two sensors of a wearable silent speech device, as discussed herein.

The first sensor data 501A is passed to a silent speech machine learning model 502. In some examples, the first sensor data is recorded from a sensor which records data when a user is speaking silently or very quietly, such as an EMG sensor, or IMU sensor, among other sensors as discussed herein. The first sensor data 501A is processed by the silent speech machine learning model 502 to determine silent speech machine learning model output 503. In the example of FIG. 5B, the silent speech machine learning model output 503 is in the same format as the second sensor data 501B. For example, the silent speech machine learning model output 503 may be an audio signal indicative of words or phrases spoken by the user, and the second sensor data is an audio recording of words or phrases spoken by the user.

The silent speech machine learning model output 503 and the second sensor data 501B are passed to training and calibration module 506. In some examples, the training and calibration module 506 may determine whether calibration is to be performed, as described herein. In some examples, the training and calibration module 506 may compare the outputs of the silent speech machine learning model 502 and the second sensor data 501B and determine a quality metric based on the difference between the data, as described herein. In some examples, the training and calibration module 506 may determine the silent speech machine learning model is to be adjusted in response to determining the quality metric value is below a threshold quality metric value.

In some examples the training and calibration module 506 may tag the silent speech machine learning model output 503 with associated data of the second sensor data 501B, for adjusting of the silent speech machine learning model 502. In the example of FIG. 5B, tagging the silent speech machine learning model output 503 may involve aligning the output with the second sensor data 501B. The tagged silent speech machine learning model output may be provided to the silent speech machine learning model to determine model adjustments 507, which are implemented in the silent speech machine learning model 502.

In some examples, the training and calibration module 506 may determine a loss function value based on the silent speech machine learning model output 503 and the second sensor data 501B, as described herein. In some examples, the loss function value is used to perform gradient descent with respect to one or more weights of the silent speech machine learning model 502. One or more weight adjustments may be determined by performing gradient descent based on the loss function value. The weight adjustments may be provided to the silent speech machine learning model 502 as model adjustments 507, as described herein.

FIG. 5C illustrates an example data flow 530 for adjusting a silent speech machine learning model, according to some embodiments of the technology described herein. As shown, recorded data 501 includes first sensor data 501A recorded from a first sensor of the wearable silent speech device, and second sensor data 501B recorded from a second sensor of the wearable silent speech device. In some examples, recorded data 501 may include data from greater than two sensors of a wearable silent speech device, as discussed herein.

First sensor data 501A and second sensor data 501B are analyzed at block 508 to determine whether the user has spoken a known word or phrase. This analysis may be performed by template matching of the first and second sensor data 501A and 501B to a known signal, using one or more machine learning models, among other techniques, as described herein. In some examples, the silent speech machine learning model 502 may determine if the user has spoken a known word or phrase. In response to determining the user has spoken a known word or phrase, the first and second sensor data 501A and 501B are passed to the silent speech machine learning model 502. In some examples, only the first sensor data 501A is passed to the silent speech machine learning model 502. In some examples, only the second sensor data 501B is passed to the silent speech machine learning model 502. In some examples, data from additional sensors may be used to record signals, which are analyzed and passed to the silent speech machine learning model 502, as described herein. In some examples, recorded data 501 is passed to silent speech machine learning model 502 for normal operations of the wearable silent speech device, as described herein, regardless of whether it is determined the user has spoken a known word or phrase.

The first and second sensor data 501A and 501B are passed to a silent speech machine learning model 502. In some examples, the first and second sensor data 501A and 501B are recorded from sensors which record data when a user is speaking silently or very quietly, such as an EMG sensor, or IMU sensor, among other sensors as discussed herein. The first and second sensor data 501A and 501B are processed by the silent speech machine learning model 502 to determine silent speech machine learning model output 503.

The silent speech machine learning model output 503 and the known signal 509 are passed to training and calibration module 506. In some examples, the training and calibration module 506 may compare the outputs of the silent speech machine learning model 502 and the known signal 509 and determine a quality metric based on the difference between the data, as described herein. In some examples a quality metric may be determined based on a confidence value associated with the silent speech machine learning model output. In some examples, the training and calibration module 506 may determine the silent speech machine learning model is to be adjusted in response to determining the quality metric value is below a threshold quality metric value.

In some examples the training and calibration module 506 may tag the silent speech machine learning model output 503 with associated data of the known signal, for adjusting of the silent speech machine learning model 502, as described herein. The tagged silent speech machine learning model output may be provided to the silent speech machine learning model to determine model adjustments 507, which are implemented in the silent speech machine learning model 502.

In some examples, the training and calibration module 506 may determine a loss function value based on the silent speech machine learning model output 503 and the known signal 509, as described herein. In some examples, the loss function value is used to perform gradient descent with respect to one or more weights of the silent speech machine learning model 502. One or more weight adjustments may be determined by performing gradient descent based on the loss function value. The weight adjustments may be provided to the silent speech machine learning model 502 as model adjustments 507, as described herein.

FIG. 5D illustrates an example data flow 540 for adjusting a silent speech machine learning model, according to some embodiments of the technology described herein. As shown, recorded data 501 includes first sensor data 501A recorded from a first sensor of the wearable silent speech device, and second sensor data 501B recorded from a second sensor of the wearable silent speech device. In some examples, the data flow of FIG. 5D may be performed using data recorded from a single sensor or from greater than two sensors. In some examples, recorded data 501 may include data from greater than two sensors of a wearable silent speech device, as discussed herein.

The recorded data 501 is passed to silent speech machine learning model 510. As shown, silent speech machine learning model 510 is an autoregressive model, which additionally receives prior first sensor data 511A and prior second sensor data 511B. In some examples, the prior sensor data may be determined from the recorded data 501. For example, the prior sensor data 511 may be a subset of the recorded data 501, such as the first 3 seconds of the recorded data, the first 4 seconds of the recorded data, the first 5 seconds of recorded data, or the first 5-10 seconds of recorded data, among other time intervals. In some examples, the prior sensor data 511 may be determined as a subset of previously recorded sensor data. For example, the prior sensor data may be data recorded at a time period before the recorded data, for example, data recorded 30 seconds before the recorded data, data recorded 1 minute before the recorded data, data recorded 2 minutes before the recorded data, data recorded 5 minutes before the recorded data, or data recorded greater than 5 minutes before the recorded data. The prior sensor data 511 may be prepended to the first and second sensor data 501A and 501B before being passed to autoregressive machine learning model 510, and being encoded at signal encoding 512.

The signal encoding 512 may encode a representation of one or more features of the sensor data 501. The features may be based on one or more aspects of the user of the wearable silent speech device. The features may be encoded based on the prior sensor data 511, the recorded sensor data 501, a subset of the recorded sensor data 501, or both the prior sensor data 511 and the recorded sensor data.

The encoded data 513 may be passed to signal decoding 514. In some examples, during signal decoding 514, the model may determine model adjustments 515 based on the encoded data. In some examples, the prior sensor data 511 may be used to determine one or more parameters of the model which are adjusted with model adjustments 515. In some examples, the one or more parameters of the model which are to be adjusted are based on the encoded representation of the recorded sensor data 501.

In some examples, the model is calibrated with model adjustments 515, determined based on the encoded data 513, before generating silent speech machine learning model output 516. For example, calibrating the silent speech machine learning model 510 may involve conditioning the model based on the prior data 511 and the conditioned model may then be used to analyze the recorded sensor data 501. In some examples, the model is calibrated with adjustments 515 after generating silent speech machine learning model output 516. In some examples, the model is continuously calibrated with model adjustments. In some examples, as the model is continuously adjusted, the model adjustments become more specific. For example, initial model adjustments 515 may be coarse adjustments to the model which involve adjusting features or weights of the model by large magnitudes, while later model adjustments are finer adjustments to the model and involve adjusting the features or weights of the model with smaller magnitude changes. In some examples, the model is provided with past outputs, which are used in the conditioning of the model.

In some embodiments, a silent speech machine learning model may be adjusted based on a data embedding generated from speech data recorded from sensors of a wearable silent speech device. The speech data may be sent to a network or model, other than the silent speech machine learning model, where a data embedding is generated. The data embedding may include latent information related to the recorded speech signals. The data embedding may then be sent to the silent speech machine learning model, which may undergo one or more adjustments based on the data embedding. In some examples, calibration of the silent speech machine learning model may be performed based on the data embedding. In some examples, training of the silent speech machine learning model may be performed based on the data embedding. In some examples, the silent speech machine learning model may be adjusted based on a received data embedding after a user of the wearable silent speech device has finished using the device. In such examples, the data embedding may be generated based on speech signals recorded when the device was in use. In some examples, the silent speech machine learning model may be adjusted based on a received data embedding while the wearable silent speech device is in use.

In some examples, different types of machine learning models may be adjusted based on prior sensor data or a subset of recorded data, similar to the autoregressive model discussed with regard to FIG. 5D. For example, the silent speech machine learning model may be a continuous flow matching model which may be adjusted based on a masked infilling technique, in which a known signal is provided to the model and k % of the samples of the known signal are masked from the model. The model may then be adjusted to predict the masked samples based on the known values for the masked samples. The model may be adjusted until a loss function representing the difference between the known values and the masked samples is below a threshold value. The continuous flow matching model may then be used to predict signals associated with the words or phrases spoken by a user from signals recorded from one or more sensors of a wearable silent speech device. In some examples, the silent speech machine learning model may be a flow matching or diffusion model, and is provided with signals from one or more sensors of the wearable silent speech device and a reference signal in the desired output format. For example, the reference signal may be an audio signal in applications where the desired output is an audio output from silent speech or the reference signal may be a transcription of silent speech signals where the desired output is a transcription of silent speech signals. In some examples, a transcription may be provided with associated silent speech signals. The machine learning model is conditioned using the reference signals, and outputs a signal in the desired format based on the input signals recorded from the one or more sensors of the wearable silent speech device.

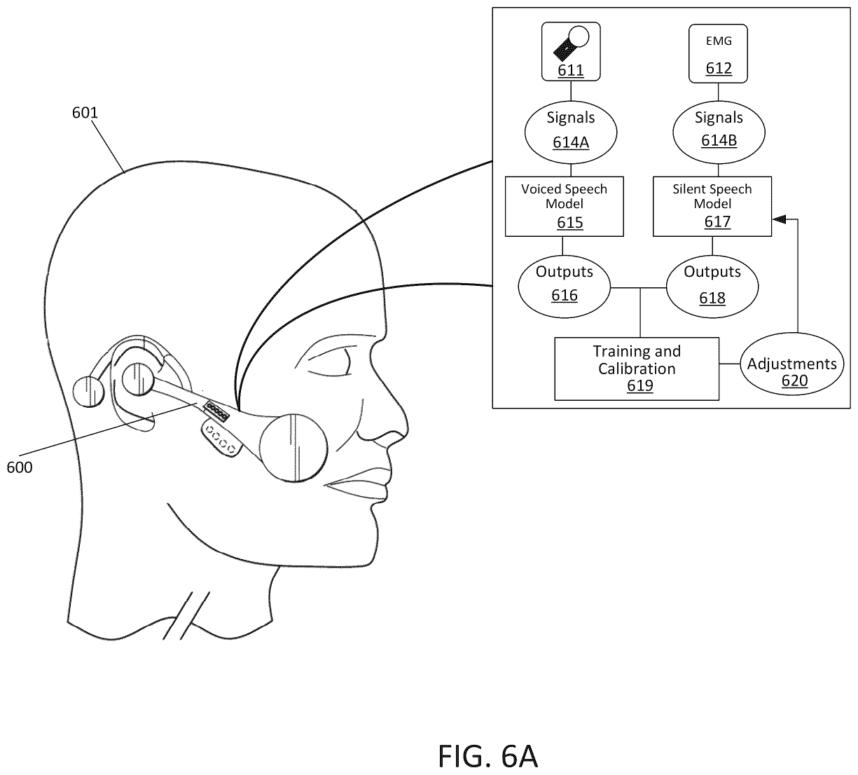

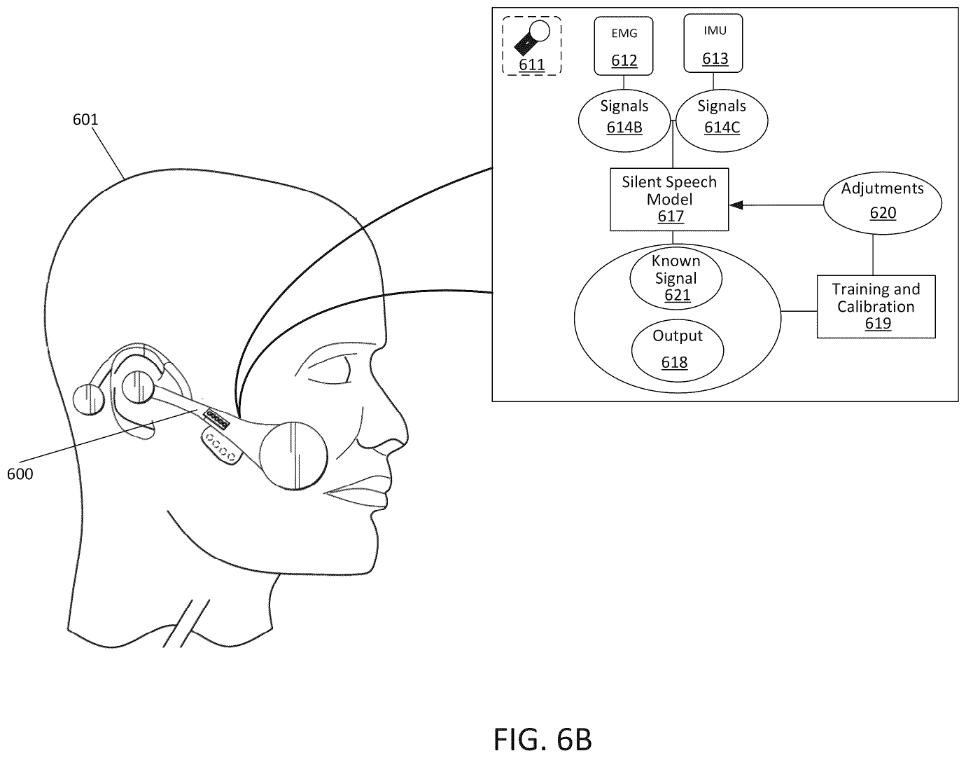

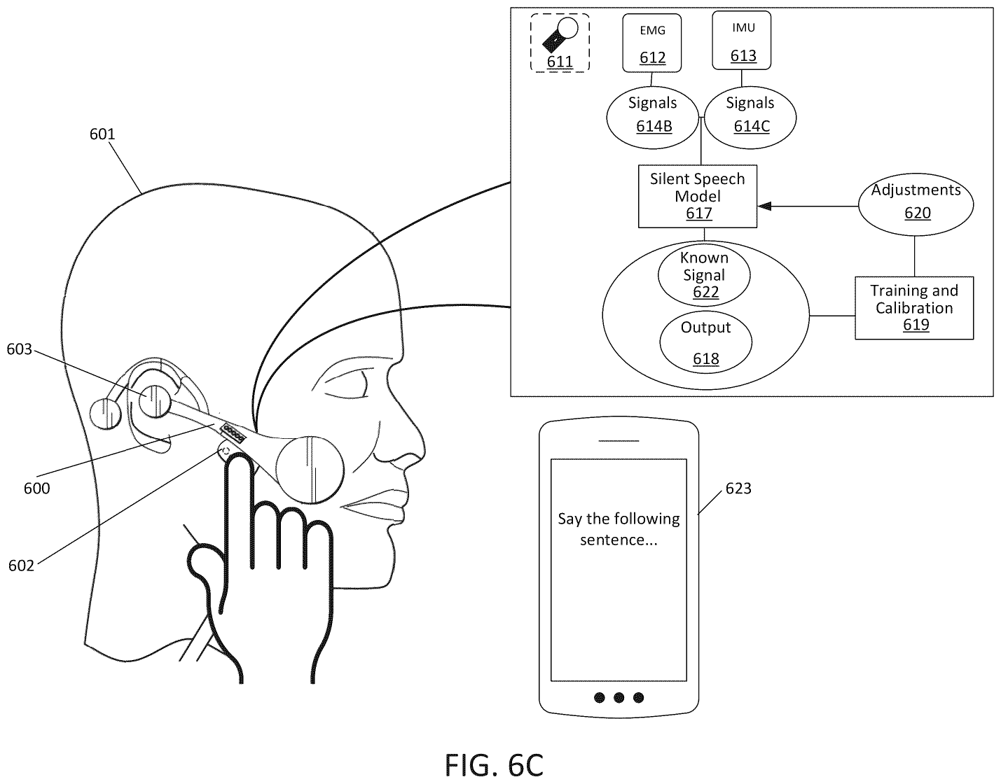

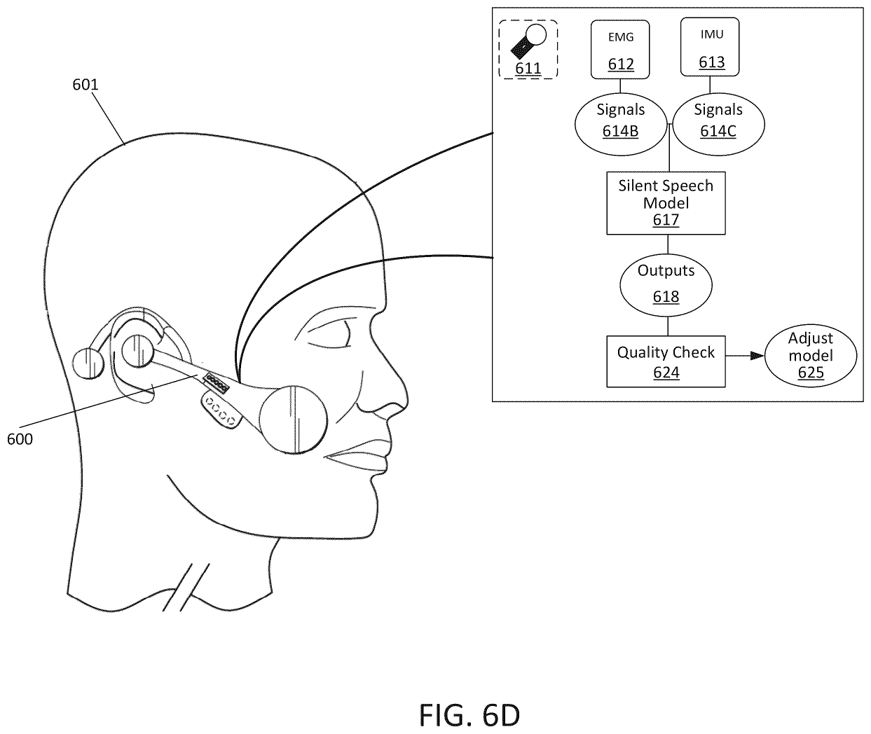

FIG. 6A provides an example of performing calibration of a silent speech machine learning model in response to a user speaking out loud, according to some embodiments of the technology described herein. In the example of FIG. 6A, the wearable silent speech device 600 may determine that the silent speech machine learning model 617 is to be adjusted in response to the user 601 speaking out loud. The wearable silent speech device 600 may determine the user 601 is speaking out loud when audio data is recorded from the microphone 611 and signals from the EMG sensor 612. In some examples, data may be recorded from additional sensors of the wearable silent speech device 600, as described herein. In some examples, voice data, such as audio recordings from a microphone, may be more accurately analyzed using a machine learning model than silent speech data such as EMG sensor recordings. In some examples, voiced data may be used as ground truth data, and is not processed by a machine learning model, when the silent speech machine learning model 617 is configured to generate an audio signal. As shown, signals recorded from the microphone 611 and the EMG sensor 612 may be passed to respective machine learning models. As shown signals 614A recorded from the microphone 611 are passed to a voiced speech machine learning model 615 and signals 614B recorded from the EMG sensor 612 are passed to a silent speech machine learning model 617. The voiced speech machine learning model outputs 616 and silent speech machine learning model outputs 618 are then compared at a training and calibration module 619, as described herein. The training and calibration module 619 may process the outputs 616, 618 of the machine learning models 615, 617 as described herein, and generate model adjustments 620 for the silent speech machine learning model 617. The model adjustments 620 may be passed to the silent speech machine learning model 617 and the model may be adjusted accordingly.

FIG. 6B provides an example of performing calibration of a silent speech machine learning model 617 in response to a user 601 speaking one or more known words, according to some embodiments of the technology described herein. In the example of FIG. 6B, the user 601 has spoken one or more known words or phrases. As shown the wearable silent speech device 600 is operating in a silent speech mode where sensors which capture silent speech signals are activated, including the EMG sensor 612 and IMU sensor 613. In some examples, sensors that capture voice speech may be deactivated in a silent speech mode, such as the microphone 611. In some examples, the process of FIG. 6B may be performed with all sensors of the wearable silent speech device 600 activated.

Signals recorded by the EMG and IMU sensor, 614B and 614C, are sent to the silent speech machine learning model 617, which may process the signals to determine whether the user has spoken one or more known words or phrases. In some examples, it may be determined whether the user has spoken one or more known words or phrases by analyzing the recorded signals 614B, 614C using a technique other than analysis by the silent speech model 617, such as template matching, as described herein.

As shown, the silent speech machine learning model 617 has determined that the user has spoken a known word or phrase, and, in response the output 618 of the silent speech machine learning model and signal associated with the known word or phrase 621 are passed to training and calibration module 619 which may compare the output 618 and known signal 621 and determine adjustments 620, as described herein. The silent speech machine learning model may then be adjusted, as described herein.

FIG. 6C provides an example of performing calibration of a silent speech machine learning model based on a user initiated calibration, according to some embodiments of the technology described herein. In the example of FIG. 6C, the user 601 has initiated calibration of the wearable silent speech device 600, which may involve adjusting the silent speech machine learning model 617. In some examples, the user 601 may initiate a calibration of the wearable silent speech device 600 when the device is not performing to the user's expectations. In some examples, the user may initiate the calibration when turning on the wearable silent speech device. As shown, the wearable silent speech device 600 is in a silent speech mode, however, calibration of the wearable silent speech device 600 may be performed in any mode of the wearable silent speech device, using any sensors of the wearable silent speech device, as described herein. The user 601 may initiate calibration of the wearable silent speech device 600 by pressing one or more buttons of the wearable silent speech device 600, such as buttons of input 602. In some examples, after pressing one or more buttons with the wearable silent speech device 600, the user may be prompted, via an external device 623, to say a known sentence. In some examples, an external device is not used for providing a known sentence. In some examples, the user may be provided a known sentence to speak via audio played through a speaker 603 of the wearable silent speech device. In some examples, calibration may be performed based on a sentence known to the user. The user may then silently speak, speak out loud, or whisper the known sentence. Signals recorded from the user 601 using the EMG sensor 612 and IMU sensor 613 are then passed to the silent speech machine learning model 617. In some examples, signals from additional sensors of the wearable silent speech device 600 may be used to record signals for calibration of the silent speech machine learning model, as described herein. The output 618 and known signal 622 may be passed to training and calibration module 619 which may determine adjustments for the silent speech machine learning model, as described herein.

FIG. 6D is an example of performing calibration of a silent speech machine learning model in response to poor performance of the model, in accordance with aspects of the technology described herein. In the example of FIG. 6D a silent speech machine learning model 617 is adjusted based on poor performance of the model. Performance of the silent speech machine learning model 617 may be determined as described herein. For example, a quality metric of the predictions of the silent speech machine learning model may be determined continuously as a user is using the wearable silent speech device. Examples of quality metrics include inaccuracy of the predictions, an error rate of the predictions, a confidence score of the predictions, a rate of correction performed on transcriptions, among other quality metrics. In the example of FIG. 6D the silent speech machine learning model output 618 may be analyzed to determine a quality metric based on a confidence score of the predictions. As shown the wearable sound speech device is operating in a silent speech mode and signals from the EMG sensor and IMU sensor are passed to the silent speech machine learning model 617. Outputs 618 of the silent speech machine learning model are analyzed at the quality check box 624 to determine the quality metric associated with the outputs. If it is determined that the quality metric is below a threshold quality metric value, then calibration of the silent speech machine learning model is performed, as described herein, as shown by adjust model 625.

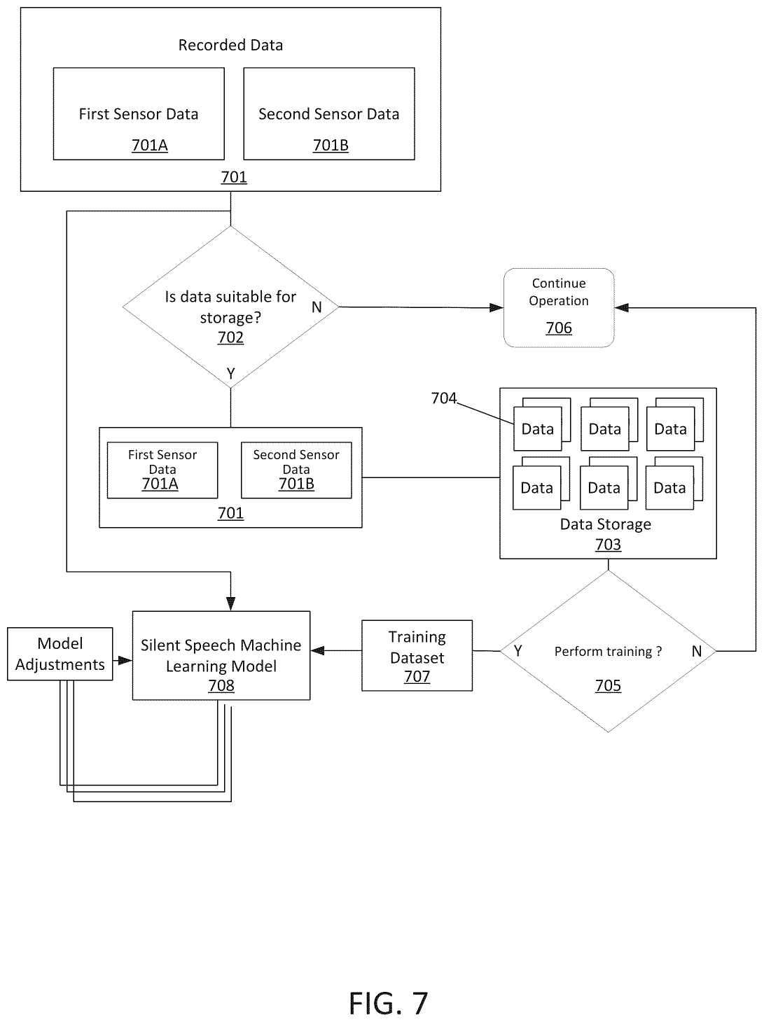

FIG. 7 illustrates an example of a process for performing training of a silent speech machine learning mode, according to some embodiments of the technology described herein.

As shown, data 701 is recorded from one or more sensors of the wearable silent speech device. The recorded data includes first sensor data 701A and second sensor data 701B, however the recorded data may include data from any number of sensors of the silent speech device, as described herein.

The recorded data 701 may be analyzed to determine whether the data is suitable for storage at block 702. In some examples data is suitable for storage when the data includes data from two or more sensors of the wearable silent speech device. In some examples, data is suitable for storage when the data includes voiced speech data recorded by a microphone and data recorded by at least one other sensor of the wearable silent speech device. In some examples, data is suitable for storage when the data indicates the user has spoken a known word or phrase. In some examples, the known word or phrase may be stored with the data. In some examples, data is suitable for storage when a quality metric of the data is above a threshold level. In some examples, data is suitable for storage when it is determined the data is associated with the user interacting with the wearable silent speech device. In some examples, data is suitable for storage when it is determined that the user is not interacting with the wearable silent speech device. When it is determined that the recorded data is suitable for storage, the data is sent to storage 703.

When it is determined recorded data 701 is not suitable for storage, the operation of the wearable silent speech device may be continued at block 706. Operation of the wearable silent speech device may include communication, transcription, and control functions, among other functions, as described herein. Recorded data 701 may be passed to silent speech machine learning model 708 as a part of operations of the wearable silent speech device, when the data is or is not suitable for storage.

The storage 703 may be non-volatile storage. In some examples, the storage 703 is located within the wearable silent speech device. In some examples, the storage 703 is located on an external device, as described herein, and the recorded data is transmitted to the external device from the wearable silent speech device. In some examples, the storage 703 is located on a server, as described herein, and the recorded data is transmitted to the server directly from the wearable silent speech device or is transmitted from the wearable silent speech device to an external device and from the external to the server.

The storage includes additional data 704. In some examples, the additional data 704 was previously recorded by the wearable silent speech device from the user of the wearable silent speech device. In some examples, the additional data 704 was previously recorded using a wearable silent speech device from individuals other than the user of the wearable silent speech device of the example of FIG. 7. In some examples, the additional data 704 is simulated data, representative of a user speaking while using a wearable silent speech device.

After the recorded data 701 is stored in storage 703, it is determined whether training of the silent speech machine learning model is to be performed at block 705, as described herein. In some examples, it is determined that training is to be performed at set time periods. In some examples, it is determined that training is to be performed when a time period has passed since the last training. In some examples, it is determined that training is to be performed when the wearable silent speech device has been used for a set period without training. In some examples, it may be determined that training is to be performed when a prompt is received requesting training be performed. In some examples, the training may be prompted by an external source. In some examples, it is determined that training is to be performed when one or more actions are performed with the wearable silent speech device. In some examples, it may be determined that training is to be performed based on a quality metric of the silent speech machine learning model output.

When it is determined that training is not to be performed, the operation of the wearable silent speech device may be continued at block 706. Operation of the wearable silent speech device may include communication, transcription, calibration, and control functions, among other functions, as described herein.

After it is determined that training is to be performed, a training dataset 707, generated from data 704, which may include recorded data 701, stored within the data storage 703 is provided to the silent speech machine learning model. In some examples, the training dataset may include all data stored in data storage, and in other examples the training dataset may include a subset of the data stored in data storage.

The training dataset 707 is provided to the silent speech machine learning model 708, where multiple epochs of training are performed using the training dataset. In some examples, the training of the machine learning model 708 involves performing one or more of: gradient descent using the training dataset, regression analysis using the training dataset, stochastic gradient descent using the training dataset, or momentum training using the training dataset. The training may be performed as described herein. For example, the training dataset may include ground truth data associated with recordings which are used in setting the weights of the silent speech machine learning model. In some examples, the ground truth data may include known words or phrases spoken by the user, or a target signal format such as an audio signal. The ground truth data may be compared to associated signals or recordings within the training dataset to determine the adjustments to the machine learning model. In some examples, the training of the machine learning model may be performed until a loss function of the silent speech machine learning model is below a threshold value.

After the training of the silent speech machine learning model 708 has been performed, adjustments 709 may be provided to the silent speech machine learning model 708, and the wearable silent speech device may be used, with the adjusted silent speech machine learning model. In some examples, training of the silent speech machine learning model may be performed, while the wearable silent speech device is in use. In such examples, the previous version of the silent speech machine learning model will be used until the training is completed and after completion, the adjusted silent speech machine learning model may be used. In some examples, the recorded sensor data may be used for other processes and functions of the wearable silent speech device and be stored within data storage. For example, the recorded signals may be used for transcription, communication, calibration, and other functions of the device, in addition to storage for training of the silent speech machine learning model.

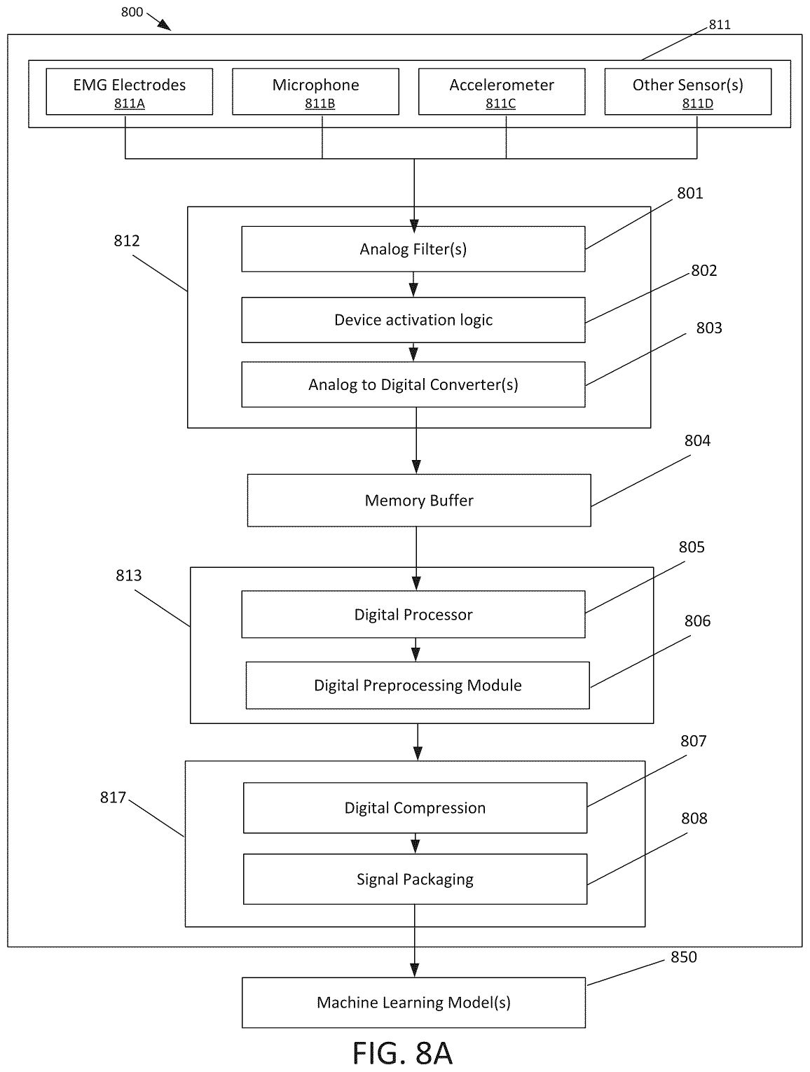

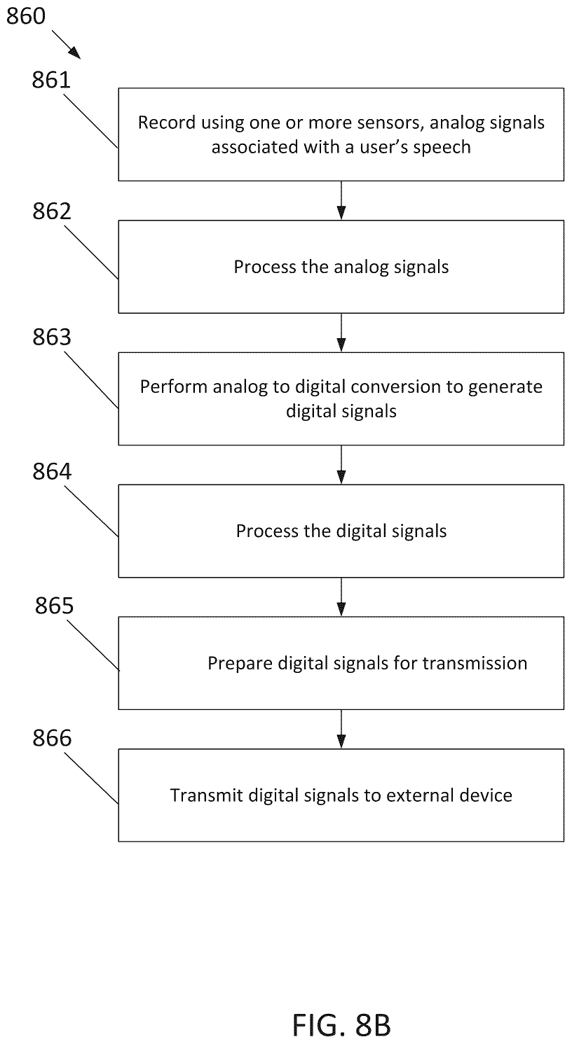

The machine learning models, including speech machine learning models and silent speech machine learning models, may be used to analyze signals recorded from a user of a wearable silent speech device during use. The machine learning model outputs may be used to control one or more functions or applications of a connected external device or to interact with a knowledge system, among other functions, as described herein. FIGS. 8A-10B provide examples of functions of wearable silent speech devices and associated machine learning models.

FIG. 8A is a scheme diagram of an example speech input device 800 capable of communicating with machine learning models 850 external to the speech input device, according to some embodiments of the technology described herein. Machine learning model 850, may include silent speech and speech machine learning models, as described herein. The machine learning model(s) 850 may be adjusted, for example with training or calibration adjustments, as described herein. In some embodiments, the speech input device 800 may be included in the wearable silent speech device 100 (FIG. 1A). In some embodiments, the speech input device 800 may include one or more sensors 811, which record signals indicating a user's speech muscle activation patterns associated with the user speaking (e.g., in a silent, voiced, or whispered speech). In non-limiting examples, the one or more sensors 811 may include one or more EMG electrodes 811A, a microphone 811B, an accelerometer 811C and/or other suitable sensors 811D. The signals collected from the sensors may be analog signals which are provided to the signal processing unit of the speech input device.

In some embodiments, the speech input device 800 may include a signal processing unit 812, one or more processors 813, and a communication interface 817. The signal processing unit 812 may include one or more analog filters 801, a device activation logic 802, and one or more analog-to-digital converters 803. The analog filters 801 may be used to improve the quality of the signals for later processing. For example, the analog filters 801 may include a high-pass filter, a low-pass filter, a bandpass filter, a moving average filter, a band stop filter, a Butterworth filter, an elliptic filter, a Bessel filter, a comb filter, and a gaussian filter, or a combination thereof. It is appreciated that the analog filters may include other suitable filters. The analog filters 801 may be implemented as a circuitry within the speech input device 800.

The device activation logic 802 may analyze the filtered signals provided from the analog filter(s) 801 to determine the presence of one or more activation signals recognized from the analog signals. For example, a user may say a particular word or phrase out loud, which is recorded by the microphone. The device activation logic 802 may recognize this word or phrase and in response will perform one or more actions. The one or more actions may include changing the mode of the device, activating one or more features of the device, and performing one or more actions. The device activation logic 802 may analyze analog filtered signals as shown, unfiltered analog signals, digital signals, filtered digital signals and/or any other signal recorded from the one or more sensors. The device activation 802 logic may operate on signals from any of the sensors, e.g., the EMG electrodes 811A, the microphone 811B, the accelerometer 811C, and any other sensors 811D in the speech input device 800. Although the device activation logic 802 is shown to be implemented in signal processing unit 812, it is appreciated that the device activation logic 802 may be implemented in any suitable component of the speech input device 800, e.g., one or more processors 813.

In some embodiments, digital converters 803 may convert analog signals to digital signals. The signals input to the analog-to-digital converters may be filtered or unfiltered signals. For example, analog signals from the one or more sensors (e.g., 811) may be directly passed to one or more analog-to-digital converters 803 without the analog filters 801. In some embodiments, there may be a respective individual analog-to-digital converter for each sensor (e.g., any of 811). The one or more analog-to-digital converters 803 may be implemented as circuitry within the speech input device 800, e.g., a chip or application specific integrated circuit (ASIC). Any suitable analog-to-digital converter circuit configuration may be used.

In some embodiments, the one or more processors 813 may perform a series of processes on the signals received from the sensors. As shown, the one or more processors 813 may process signals from the one or more sensors 811, or via the signal processing unit 812. Additionally, and/or alternatively, the speech input device 800 may include one or more memory buffers 804. The memory buffers 804 may temporarily store data as it is transferred between the signal processing unit 812 and one or more processors 813, or between any other internal units of the one or more processors 813, or between any components of the speech input device 800. The memory buffers 804 may be implemented as hardware modules or may be implemented as software programs which store the data in a particular location within a memory of the speech input device 800. The memory buffers 804 may store data including analog and/or digital signals, such as filtered signals from analog filter(s) 801, digital signals from analog-to-digital converter(s) 803, control signals from the device activation logic 802, and any other data from within the speech input device 800.

In some embodiments, the one or more processors 813 may include a digital signal processor 805 configured to perform digital signal processing on digital signals from the analog-to-digital converter(s) 803, for example, or digital data stored in the memory buffer 804. In some embodiments, digital signal processor 805 may process the digital signals and improve the quality thereof for later processes. In some embodiments, the digital signals may undergo one or more digital processing operations in the digital signal processor 805. In some embodiments, the digital processing in the digital signal processor 805 may be tailored to specific signals, e.g., signals from the EMG electrodes 811A, which may undergo specific digital processing that is different from processing executed on signals recorded from the microphone 811B. Examples of digital signal processing performed in the digital signal processor 805 include digital filtering of the signals, feature extraction, Fourier analysis of signals, Z-plane analysis, and/or any other suitable digital processing techniques.

In some examples, the digital signal processor 805 may include one or more layers of a neural network and/or a machine learning model maintained by the speech input device to generate digital signal vector(s). Additionally, and/or alternatively, the one or more processors 813 may include a digital preprocessing component 806 configured to perform one or more preprocessing operations, e.g., normalization of data, cropping of data, sizing of data, reshaping of data, and/or other suitable preprocessing actions.

In some embodiments, the communication interface 817 may be configured to receive signals from other units, e.g., 811, 812, 813, and prepare data for further processing. In some embodiments, the communication interface 817 may include a digital compressor 807 configured to compress the received signals and a signal packets generator 808 configured to perform signal packaging for transmission. In some embodiments, the signals received at the communication interface 817 may undergo digital compression at the digital compressor 807 and the compressed data from digital compressor 807 may be packaged for transmission. In non-limiting examples, digital compression may be performed at digital compressor 807 on one or more signals in order to reduce the amount of data transmitted by the speech input device. Digital compression performed at digital compressor 807 may use any suitable techniques, e.g., lossy and lossless compression techniques.

In some embodiments, signal packaging may be performed at signal packets generator 808 to format (e.g., packetize) data for transmission according to a particular transmission modality. For example, a signal may be packetized with additional information to form a complete Bluetooth packet for transmission to an external Bluetooth device. In the example shown in FIG. 8A, the packetized signal may be sent to an external device having a machine learning model 850.

FIG. 8B is a flow diagram of an example process 860 which may be performed by a speech input device such as speech input device 800 shown in FIG. 8A, according to some embodiments of the technology described herein. In some embodiments, process 860 may be performed by one or more components in the speech input device 800 (FIG. 8A) to capture sensor data when the user is speaking and process the sensor data before transmitting to an external device. In some embodiments, method 860 may start with capturing, at one or more sensors (e.g., 811 in FIG. 8A), speech signals from a user associated with the user's speech, at act 861. In some embodiments, the speech signals captured from the sensors may be analog signals. Method 860 may further include processing the captured analog signals at act 862. In some examples, act 862 may be performed at signal processing unit 812 (FIG. 8A) and may include various processing operations, e.g., filtering, feature extraction, device activation, and machine learning processing, among other techniques as described above and further herein.

With further reference to FIG. 8B, method 860 may include performing analog-to-digital conversion to generate digital signals, at act 863. In some examples, act 863 may be performed at analog-to-digital converter(s) (e.g., 803 in FIG. 8A). Method 860 may further include processing the digital signals, at act 864. For example, act 864 may be performed at digital signal processor 805, and optionally, digital preprocessing component 806 (FIG. 8A). For example, act 864 may include digital filtering of the signals, feature extraction, Fourier analysis of signals, machine learning processing and Z-plane analysis, among other processing techniques as described above and further herein.

With further reference to FIG. 8B, method 860 may further include preparing digital signals for transmission, at act 865. In some embodiments, act 865 may be performed at communication interface 817 (FIG. 8A). For example, act 865 may include preprocessing signals, compressing signals and packetizing data as discussed above and further herein. Method 860 may also include transmitting the signals from act 865 to an external device, at act 866. The signals may be transmitted using any suitable protocol, as discussed herein.

In some embodiments, the signals transmitted from the speech input device 800 to the external device (e.g., 850 in FIG. 8A) may include sensor data associated with a user's speech (e.g., silent speech), or the processed sensor data. The external device may include a speech model configured to convert the sensor data (or processed sensor data) to text or encoded features for use with any suitable system, where the encoded features may include information about the uncertainty of the text. Thus, the combination of the speech input device and the external device enables a wide range of systems and applications that can utilize the speech model. In non-limiting examples, the external device may be a computer, a laptop, or a mobile phone that includes a speech model, and is capable of communicating with speech input device (e.g., 800) to receive the sensor data associated with a user's speech, where the speech model is also configured to convert the sensor data to text or encoded features. The computer, laptop, or the mobile phone may implement any application to take one or more actions. For example, the computer, laptop, or the mobile phone may implement a user interaction system, which receives text prompt or encoded features from the speech model to take one or more actions. The user interaction system may be implemented in the computer to interact with a knowledge system by providing the received text prompt or encoded features from the speech model to the knowledge system and cause the knowledge system to take the one or more actions. It is appreciated that any other suitable systems may be enabled by the speech input device.

It is appreciated that the various processes as discussed with acts in method 860 may not be all performed or may be performed in any suitable combination or order. Each signal as captured at the one or more sensors (e.g., 811) may have associated processing operations that may be tailored to that particular signal. Different types of signals may be processed in a series of respective different operations. For example, signals from the EMG electrodes may undergo all operations in method 860 whereas signals from the microphone may only undergo analog to digital conversion at act 863 and digital processing at act 864. In some embodiments, the processing performed at each of the processing operations of in a series of processing operations in method 860 may also be different for each signal received from the sensor(s). For example, analog filters used by act 862 may include a high-pass filter for signals received from the microphone and include a bandpass filter for signals received from the EMG electrodes.

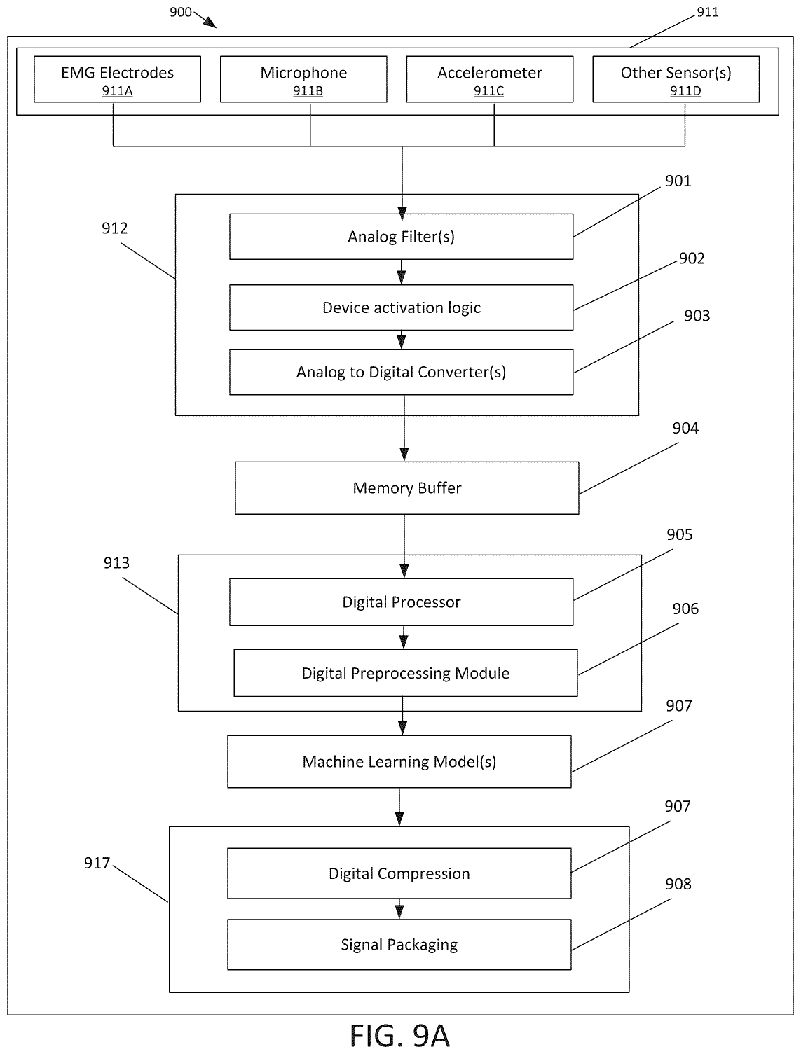

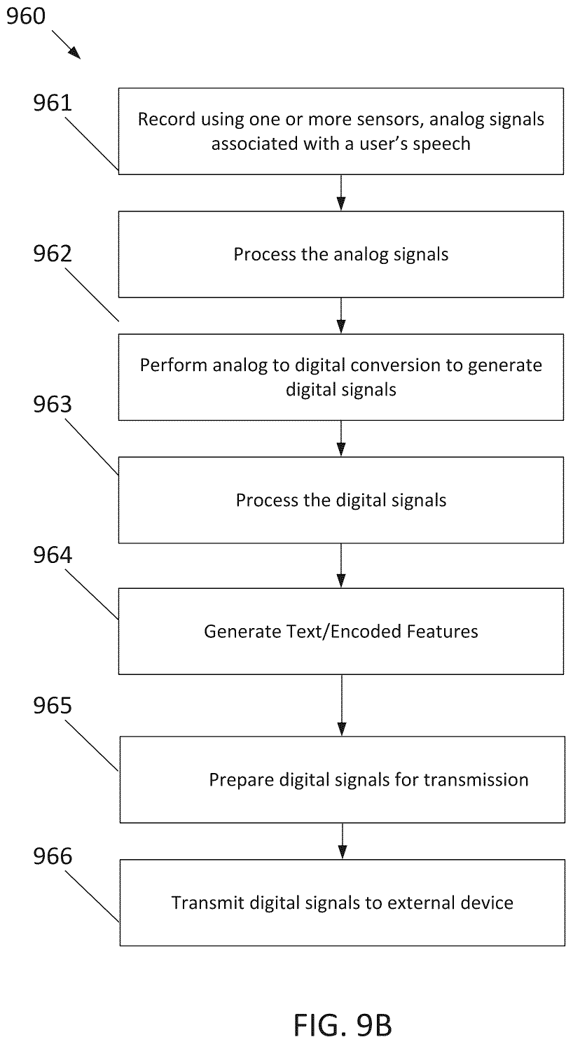

FIG. 9A is a scheme diagram of an example speech input device 900 including a silent speech model, according to some embodiments of the technology described herein. In some embodiments, speech input device 900 may have a similar configuration as speech input device 800 (FIG. 8A) with a difference being that speech input device 900 in FIG. 9A includes an embedded speech model 915, rather than the speech model being external to the speech input device as in FIG. 8A. The machine learning model(s) 915 may include a speech machine learning model or a silent speech machine learning model, as described herein. The machine learning model(s) 915 may be adjusted, for example with training or calibration adjustments, as described herein. Thus, the numerals 800's in FIG. 8A and numerals 900's in FIG. 9A may correspond to similar components when the last two digits are the same. For example, 911 in FIG. 9A may correspond to one or more sensors 811 in FIG. 8A for capturing electrical signals indicating the user's speech muscle activation patterns or other measurements when the user is speaking (e.g., in a voiced, silent, or whisper speech). Similarly, 912 in FIG. 9A may correspond to signal processing unit 812 in FIG. 8A.

As shown in FIG. 9A, speech input device 900 may additionally include machine learning model(s) 915 configured to convert the digital signals from one or more processors 913 to text or encoded features. The machine learning model(s) 915 may include silent speech and speech machine learning models, as described herein. With further reference to FIG. 9A, speech model 915 may provide the text or encoded features to the communication interface 917 for transmitting to an external device. In some embodiments, the communication interface 917 may transmit the compressed/packetized text or encoded features to an application on the external device via a communication link such as a wired connection or a wireless connection.

FIG. 9B is a flow diagram of an example process 960 including the use of a silent speech model, where the process may be performed by a speech input device, e.g., 900 (FIG. 9A) according to some embodiments of the technology described herein. Various acts in process 960 may correspond to acts with the numerals alike in process 860 in FIG. 8B. For example, method 960 may be similar to method 860 (FIG. 8B), with a difference being that method 960 may generate text or encoded features at act 967, where act 967 may be performed using a speech model (e.g., 915 in FIG. 9A). Subsequent to generating the text or encoded features, method 960 may prepare the output of the speech model (e.g., compressing, packetizing) at act 965, and transmit the output to the external device, at act 966.

As similar to FIGS. 8A and 8B, speech input device 900 (FIG. 9A), with the combination of an external device may enable a wide range of systems and applications in a similar manner as with speech input device 800 (FIG. 8A). In non-limiting examples, the external device may be a computer, a laptop, or a mobile phone that is capable of communicating with speech input device (e.g., 900) to receive text or encoded features associated with the user's speech, where the text prompt or encoded features are generated by the speech model in the speech input device, using the sensor data captured at the speech input device. The external device may use the received text or encoded features to enable any application. For example, the application may be an interaction system, which receives the text prompt or encoded features from the speech model and provide the text prompt or encoded features to a knowledge system to take one or more actions.

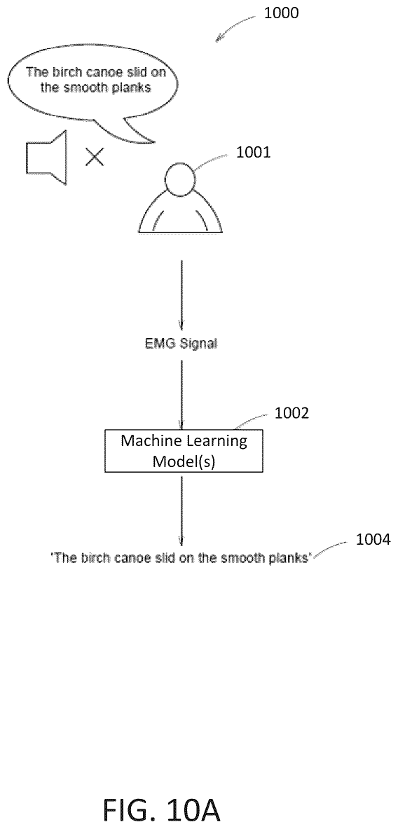

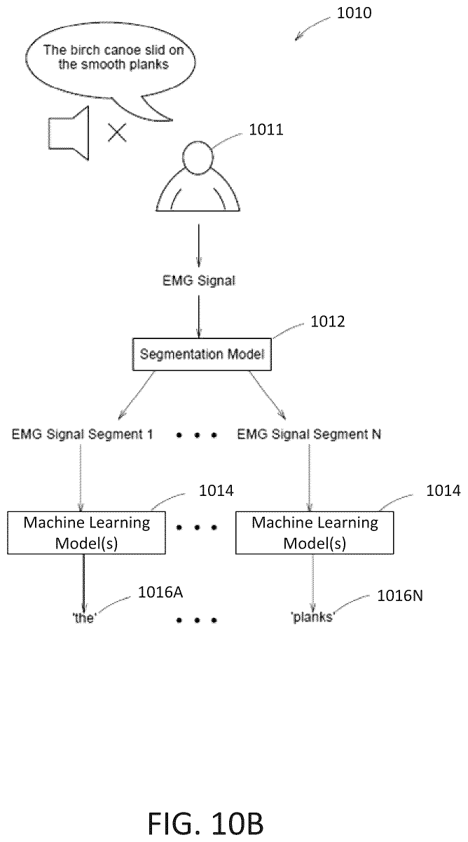

FIG. 10A is a scheme diagram of a machine learning model configured to decode speech to predict text or encoded features using EMG signals, according to some embodiments of the technology described herein. The machine learning model may operate in a process flow, for example process flow 1000. The machine learning model 1002 may include a silent speech machine learning model or a speech machine learning model, as described herein. In some embodiments, the machine learning model 1002 may be trained and installed in a wearable device, such as wearable device 100 of FIG. 1A or any other wearable device discussed herein. Alternatively, the machine learning model 1002 may be installed in an external as described herein. When deployed (for inference), the machine learning model 1002 may be configured to receive sensor data indicative of the user's 1001 speech muscle activation patterns (e.g., EMG signals) associated with the user's speech (voiced or silent) and use the sensor data to predict text or encoded features. The machine learning model 1002 may be a speech machine learning model or a silent speech machine learning model, as described herein. The machine learning model(s) 1002 may be adjusted, for example with training or calibration adjustments, as described herein. As shown in FIG. 10A, the user speaks silently “The birch canoe slid on the smooth planks” 1004. The machine learning model 1002 receives the EMG signals associated with the user's speech 1004, where the EMG signals indicate the speech muscle activation patterns as discussed above and further herein. The machine learning model 1002 outputs the text “The birch canoe slid on the smooth planks.”

In some embodiments, the sensor data indicating the user's speech muscle activation patterns, e.g., EMG signals, may be collected using a wearable device. The machine learning model 1002 may be trained to use the sensor data to predict text or encoded features. Although it is shown that the EMG signals is associated with the user speaking silently, it is appreciated that the EMG signals may also be associated with the user speaking loudly, or in whisper, and may be used train the speech model to predict the text or encoded features, as described herein.

FIG. 10B is a scheme diagram of a machine learning model 1014 configured to decode speech to predict text or encoded features using EMG signals and segmentation of the EMG signals, according to some embodiments of the technology described herein. As shown, FIG. 10B is similar to FIG. 10A with a difference in that the signals indicating the user's 1011 speech muscle activation patterns (e.g., EMG signals) are segmented by a segmentation model 1012 before being provided to the machine learning model 1014. In the example process flow 1010 shown, the EMG signals are segmented into a number of segments (e.g., 1, 2, . . . , N). These EMG signal segments are provided to the machine learning model 1014, which is configured to output the text corresponding to each of the EMG signal segments. In some embodiments, the EMG signals are segmented by word, for example, the speech “The birch canoe slid on the smooth planks” is segmented by eight segments each corresponding to a respective word in the speech. As shown, the machine learning model 1014 may output eight words 1016A- . . . 1016N each corresponding to a respective EMG signal segment. Although it is shown that segmentation model 1012 segments the EMG signals by word, it is appreciated that the segmentation model may also be trained to segment the EMG signals in any other suitable manner, where each segment may correspond to a phoneme, a syllabus, a phrase, or any other suitable segment unit. Accordingly, the machine learning model 1014 may be trained to predict text that corresponds to a signal segment (e.g., EMG signal segment), where a segment may correspond to a segmentation unit, e.g., a sentence, a phrase, a word, a syllable etc. In some embodiments, training a speech model (e.g., 1014) for predicting text segments may include generating segmented training data, which may be used for training machine learning models, as described herein. The machine learning model 1002 may be a speech machine learning model or a silent speech machine learning model, as described herein. The machine learning model(s) 1002 may be adjusted, for example with training or calibration adjustments, as described herein.

It should be appreciated that one or more aspects described herein may be practiced with any of the embodiments, examples, and implementations described in U.S. patent application Ser. No. 18/338,827, titled “WEARABLE SILENT SPEECH DEVICE, SYSTEMS, AND METHODS”, filed Jun. 21, 2023, the content of which is an integral part of this application and is incorporated by reference in its entirety.

Having thus described several aspects of at least one embodiment of the technology described herein, it is to be appreciated that various alterations, modifications, and improvements will readily occur to those skilled in the art. Such alterations, modifications, and improvements are intended to be part of this disclosure and are intended to be within the spirit and scope of disclosure. Further, though advantages of the technology described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software, or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the technology described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the technology as described above. A computer-readable storage medium includes any computer memory configured to store software, for example, the memory of any computing device such as a smart phone, a laptop, a desktop, a rack-mounted computer, or a server (e.g., a server storing software distributed by downloading over a network, such as an app store)). As used herein, the term “computer-readable storage medium” encompasses only a non-transitory computer-readable medium that can be considered to be a manufacture (i.e., article of manufacture) or a machine. Alternatively, or additionally, aspects of the technology described herein may be embodied as a computer readable medium other than a computer-readable storage medium, such as a propagating signal.

The terms “program” or “software” are used herein in a generic sense to refer to any type of computer code or set of processor-executable instructions that can be employed to program a computer or other processor to implement various aspects of the technology as described above. Additionally, it should be appreciated that according to one aspect of this embodiment, one or more computer programs that when executed perform methods of the technology described herein need not reside on a single computer or processor but may be distributed in a modular fashion among a number of different computers or processors to implement various aspects of the technology described herein.

Computer-executable instructions may be in many forms, such as program modules, executed by one or more computers or other devices. Generally, program modules include routines, programs, objects, modules, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys the relationship between the fields. However, any suitable mechanism may be used to establish a relationship between information in fields of a data structure, including through the use of pointers, tags or other mechanisms that establish relationship between data elements.

Various aspects of the technology described herein may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the technology described herein may be embodied as a method, of which examples are provided herein including with reference to FIGS. 4D, 8B, and 9B. The acts performed as part of any of the methods may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately” and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method comprising acts of:
recording speech signals from a user, using a first sensor and a second sensor of a wearable silent speech device;
providing for a silent speech machine learning model for use with the wearable silent speech device;
determining whether the silent speech machine learning model is to be adjusted; and
in response to determining the silent speech machine learning model is to be adjusted, adjusting the silent speech machine learning model based on at least the speech signals recorded using the first sensor and the second sensor.

2. The method of claim 1, further comprising:
determining a subset of the recorded speech signals, wherein adjusting the silent speech machine learning model comprises:
providing the subset of the recorded speech signals to the silent speech machine learning model;
conditioning the silent speech machine learning model based on the subset of the recorded speech signals; and
processing, using the conditioned silent speech machine learning model, the recorded speech signals to generate a representation of one or more words spoken by the user.

3. The method of claim 1, further comprising:
storing the recorded speech signals in non-volatile storage, the non-volatile storage storing historic speech signals recorded by the wearable silent speech device; and
wherein adjusting the silent speech model comprises training the silent speech machine learning model based on the speech signals recorded using the first sensor and the second sensor and the historic speech signals.

4. The method of claim 3, wherein training the silent speech machine learning model comprises performing a series of gradient steps based on a comparison of an output of the silent speech machine learning model to ground truth data.

5. The method of claim 4, wherein the non-volatile storage stores the ground truth data associated with the historic speech signals.

6. The method of claim 1, wherein the first sensor is an EMG sensor and the second sensor is a microphone.

7. The method of claim 1, wherein the determining comprises determining whether the recorded speech signals are suitable for use in adjusting the silent speech machine learning model, and determining the silent speech machine learning model is to be adjusted in response to determining the recorded speech signals are suitable for use in adjusting the silent speech machine learning model.

8. The method of claim 7, wherein determining whether the recorded speech signals are suitable comprises determining, based on the speech signals, whether the user is speaking out loud and determining the recorded speech signals are suitable in response to determining the user is speaking out loud.

9. The method of claim 7, wherein determining whether the recorded speech signals are suitable comprises determining, based on the speech signals, a level of background noise and determining the recorded speech signals are suitable in response to determining the level of background noise is below a threshold level.

10. The method of claim 1, wherein determining whether the silent speech machine learning model is to be adjusted comprises determining whether the silent speech machine learning model requires user onboarding, and in response to determining the silent speech machine learning model requires user onboarding, prompting the user to speak one or more words or phrases, wherein the speech signals are recorded after the prompting.

11. The method of claim 1, wherein determining whether the silent speech machine learning model is to be adjusted comprises:
determining a performance metric of the silent speech machine learning model; and
determining the silent speech machine learning model is to be adjusted in response to determining the performance metric is below a threshold level.

12. The method of claim 1, wherein determining whether the silent speech machine learning model is to be adjusted comprises determining, based on a user input, whether the silent speech machine learning model is to be adjusted.

13. The method of claim 1, further comprising determining whether the wearable silent speech device is being powered on, and in response to determining the wearable silent speech device is being powered on, prompting the user to speak one or more words or phrases, wherein the speech signals are recorded after the prompting, and it is determined that the silent speech machine learning model is to be adjusted in response to determining the wearable silent speech device is being powered on.

14. The method of claim 1, wherein determining whether the silent speech machine learning model is to be adjusted comprises determining a time since a last silent speech machine learning model adjustment, and in response to determining the time is above a threshold time, determining the silent speech machine learning model is to be adjusted.

15. The method of claim 1, further comprising:
analyzing the recorded speech signals; and
selecting a subset of the recorded speech signals, wherein the adjusting is performed using the subset of the recorded speech signals.

16. The method of claim 1, wherein adjusting the silent speech machine learning model comprises performing a gradient step of the silent speech machine learning model based on a comparison of an output of the silent speech machine learning model to ground truth data.

17. The method of claim 16, further comprising determining the ground truth data based on the recorded speech signals.

18. The method of claim 17, wherein the ground truth data is determined using a second machine learning model, different from the silent speech machine learning model.

19. A system for recognizing silent speech of a user, the system comprising:
a wearable silent speech device;
at least one computer hardware processor; and
at least one non-transitory computer-readable storage medium storing processor-executable instructions that, when executed by the at least one computer hardware processor, cause the at least one computer hardware processor to perform a method, the method comprising:
obtaining speech signals recorded from the user, using a first sensor and a second sensor of the wearable silent speech device;
providing for a silent speech machine learning model for use with the wearable silent speech device;
determining whether the silent speech machine learning model is to be adjusted; and
in response to determining the silent speech machine learning model is to be adjusted, adjusting the silent speech machine learning model based on at least the speech signals recorded using the first sensor and the second sensor.

20. At least one non-transitory computer-readable storage medium storing processor-executable instructions that, when executed by at least one computer hardware processor, cause the at least one computer hardware processor to perform a method, the method comprising:
obtaining speech signals recorded from a user, using a first sensor and a second sensor of a wearable silent speech device;
providing for a silent speech machine learning model for use with the wearable silent speech device;
determining whether the silent speech machine learning model is to be adjusted; and
in response to determining the silent speech machine learning model is to be adjusted, adjusting the silent speech machine learning model based on at least the speech signals recorded using the first sensor and the second sensor.
